(19) United States
(12) Patent Application Publication
(10) Pub. No.: US 2025/0313618 A1
(43) Pub. Date: Oct. 9, 2025

(54) Methods of treating giant cell arteritis using Interleukin-17 (IL-17) Antagonists

(71) Applicants: NOVARTIS AG, Basel (CH); ALBERT-LUDWIGS-UNIVERSITAT FREIBURG, Freiburg (DE)

(72) Inventors: Nils VENHOFF, Freinurg (DE); Jens THIEL, Graz (AT); Claudia JENTZSCH, Magdeburg (DE); Carolin LOPIAN, Nuernberg (DE); Meryl MENDELSON, East Hanover, NJ (US); John H. STONE, Concord, MA (US)

(21) Appl. No.: 18/865,343

(22) PCT Filed: May 16, 2023
(86) PCT No.: PCT/IB2023/055041

§ 371 Date: Nov. 13, 2024

Related U.S. Application Data
(60) Provisional application No. 63/342,554, filed on May 16, 2022.
(60) Provisional application No. 63/382,795, filed on Nov. 8, 2022.

(51) Int. Cl. C07K 16/24; A61K 39/00; A61P 9/00

(52) U.S. Cl. CPC C07K 16/244; A61P 9/00; A61K 2039/505; A61K 2039/545

(57) ABSTRACT

The present disclosure relates to methods for treating giant cell arteritis (GCA), using IL-17 antagonists, e.g., secukinumab. Also disclosed herein are uses of IL-17 antagonists, e.g., IL-17 antibodies, such as secukinumab, for treating GCA patients, as well as medicaments, dosing regimens, pharmaceutical formulations, dosage forms, and kits for use in the disclosed uses and methods.

### TECHNICAL FIELD

The present disclosure relates to methods of treating giant cell arteritis (GCA) using IL-17 antagonists, e.g., an IL-17 antibody or antigen-binding fragment thereof, such as secukinumab, or ixekizumab, or an IL-17 receptor antibody or antigen-binding fragment thereof, such as brodalumab.

### BACKGROUND OF THE DISCLOSURE

Giant cell arteritis (GCA) is the most common form of primary systemic vasculitis in people over the age of 50 years (Koster et al 2016). GCA is an inflammatory chronic disease with a prevalence between 24 and 278 per 100,000 in the European Union (EU) and the United States of America (USA). Typical clinical manifestations of new-onset GCA related to the inflammation of large- and medium-sized arteries are new onset of headaches, jaw claudication, scalp tenderness, and visual disturbances. Characteristic systemic manifestations include fever, malaise, weight loss, and polymyalgia (Ness et al 2013). Ischemic anterior optic neuropathy resulting in irreversible visual loss is a common and feared symptom of GCA. Therefore, prompt and effective immunosuppressive treatment is crucial in GCA (Hoffmann et al 2002, Jover et al 2001).

High dose glucocorticoids are the mainstay of GCA therapy and effectively reduce vascular inflammation (Salvarani et al 2004, Petri et al 2014). While glucocorticoids remain the mainstay of treatment, relapses are common and morbidity related to treatment frequently occurs (Koster et al 2016). High cumulative glucocorticoid doses have the major drawback of a high rate of adverse events (AEs) with more than 80% of patients suffering from serious adverse events (SAEs). High glucocorticoid sum doses are of major concern as they result in a substantial increase of infections, osteoporosis, and severe metabolic side effects. Furthermore, treatment failures occur in more than 50% of patients (Salvarani et al 2004, Petri et al 2014). There is an unmet need for immunosuppressive therapies that are able to induce long-term remission while avoiding the adverse effects of glucocorticoids (Koster et al 2016).

Studies on anti-tumor necrosis factor alpha (TNFα) therapies and azathioprine have failed to demonstrate a consequential effect while results of studies on methotrexate as glucocorticoid-sparing agents are conflicting (Petri et al 2014, De Silvia et al 1986, Hayat 2008). The GIACTA-trial (Stone et al 2017) showed that tocilizumab, an interleukin-6 receptor antagonist, (administered weekly or every other week) combined with a 26-week prednisolone taper was superior to either 26-week or 52-week prednisolone tapering plus placebo with regard to sustained glucocorticoid-free remission in patients with GCA. Nonetheless, there were some drawbacks regarding tocilizumab therapy in GCA patients which should be considered. The primary endpoint (sustained remission at Week 52) was achieved in 56% of patients in the group that received tocilizumab weekly and 53% of patients in the group that received tocilizumab every other week; therefore, almost half of patients did not achieve remission. Furthermore, high sensitivity CRP, which was chosen as part of the definition of remission in the GIACTA trial, is not reliable as an inflammation marker (for indicating disease relapses or infectious complications) in GCA patients treated with tocilizumab as CRP is suppressed under tocilizumab therapy. Moreover, a magnetic resonance imaging (MRI) follow-up of the phase II study by Reichenbach et al (2018) on the use of tocilizumab in GCA showed persistent large vessels contrast-media enhancement, which indicates ongoing large-vessel inflammation. Thus additional treatment alternatives besides glucocorticoids and tocilizumab are needed in GCA.

The cause of GCA has still not been clearly identified but it is thought that GCA occurs based on a genetic background and is triggered by unknown environmental factors that can activate and lead to maturation of dendritic cells localized in the adventitia of normal arteries. Activated dendritic cells then lead to the activation, proliferation and polarization of Th1 and Th17 cells, which produce interferon-gamma and interleukin 17 (IL-17), respectively (Samson et al 2017). Interleukin 17A (IL-17A) seems to be implicated in the pathogenesis of GCA; increased IL-17A expression in temporal artery lesions is a predictor of sustained response to glucocorticoid treatment in patients with GCA (Espigol-Frigole et al 2013, Samson et al 2012). Recent evidence suggests that there is heterogeneity of histological lesions in GCA, which is correlated with Th9 and especially Th17 (Ciccia et al 2017). Marquez et al found a novel association between polymorphisms within the IL-17A locus and GCA that supports the relevant role of Th17 cells in this vasculitis pathophysiology (Marquez et al 2014). Another study shows hyperproliferation of regulatory T-cells that overexpress the FoxP342 domain, lacking the 42. The dysfunctional FoxP342 domain is known to contribute to an enhanced Th17 differentiation and therefore IL-17A overproduction. In line with that observation, IL-17A is upregulated in active GCA patients of that study. The IL-6 receptor antagonist tocilizumab is able to reestablish the functional FoxP3 domain in regulatory T-cells leading to less IL-17A production and effective control of GCA. This observation implicates IL-17A to be an important cytokine in active GCA and its direct inhibition to be a new therapeutic target for patients suffering from active disease. (Miyabe et al 2017).

Two case reports of treating a patient with secukinumab to maintain remission of GCA have been reported GCA (Rotar et al 2018, Sammut et al 2018). However, in the case of Rotar et al., it is clear that the patient was already in remission before secukinumab treatment was initiated. In Sammut, the patient's GCA appeared to be well-controlled by high dose oral prednisolone prior to secukinumab treatment. Accordingly, therapeutically effective treatment of a patient suffering from, e.g., active, GCA with an IL-17 antagonist has not been demonstrated. Moreover, the durability of the treatment response has not been shown.

### SUMMARY OF THE DISCLOSURE

Secukinumab is a selective high-affinity fully human monoclonal antibody that neutralizes IL 17A and is approved for treating plaque psoriasis, psoriatic arthritis (PsA), and ankylosing spondylitis (AS). We have now determined that IL-17 antagonists, e.g., IL-17 antibodies, e.g., secukinumab, can be used systemically to treat giant cell arteritis (GCA).

Disclosed herein are methods, uses, pharmaceutical compositions, and kits for inducing regeneration of vessel tissue or promoting vessel repair, or reducing vessel inflammation, in a patient having GCA, comprising subcutaneously administering to a patient in need thereof about 150 mg-about 300 mg (e.g., a fixed dose of about 150 mg, a fixed dose of about 300 mg) of an IL-17 antibody or antigen-binding fragment thereof, wherein the IL-17 antibody or antigen-binding fragment thereof binds to an epitope of a human IL-17 homodimer having two mature human IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

In some embodiments of the disclosed methods, uses, pharmaceutical compositions, and kits, the patient is administered the IL-17 antibody or antigen-binding fragment thereof weekly.

In some embodiments of the disclosed methods, uses, pharmaceutical compositions, and kits, the patient is administered the IL-17 antibody or antigen-binding fragment thereof during week 0, 1, 2, 3, and 4.

In some embodiments of the disclosed methods, uses, pharmaceutical compositions, and kits, the patient is administered the IL-17 antibody or antigen-binding fragment thereof every two weeks or every 4 weeks, preferably every 4 weeks.

In some embodiments of the disclosed methods, uses, pharmaceutical compositions, and kits, the patient is administered the IL-17 antibody or antigen-binding fragment thereof for a total treatment duration of at least two months, at least 26 weeks, at least 28 weeks, at least 52 weeks, or at least 2 years.

In some embodiments of the disclosed methods, uses, pharmaceutical compositions, and kits, the patient is administered, e.g., 150 or 300 mg of, the IL-17 antibody or antigen-binding fragment thereof weekly during week 0, 1, 2, 3, and 4, and then every 4 weeks.

In some embodiments of the disclosed methods, uses, pharmaceutical compositions, and kits, the patient is administered the IL-17 antibody or antigen-binding fragment thereof during week 0, 1, 2, 3, 4, 8 and 12.

In some embodiments of the disclosed methods, uses, pharmaceutical compositions, and kits, the patient is administered the IL-17 antibody or antigen-binding fragment thereof weekly during week 0, 1, 2, 3, and 4, and then every 4 weeks thereafter, for a total treatment duration of at least 26 weeks.

In some embodiments of the disclosed methods, uses, pharmaceutical compositions, and kits, prior to treatment with the IL-17 antibody or antigen-binding fragment thereof, the patient failed to respond to, had an inadequate response to, or was intolerant to a prior GCA treatment selected from the group consisting of a corticosteroid, such as prednisone, prednisolone, or methylprednisolone, treatment with a TNF-alpha inhibitor, treatment with an IL-6 inhibitor, treatment with methotrexate, and combinations thereof.

In some embodiments of the disclosed methods, uses, pharmaceutical compositions, and kits, the patient has active GCA.

In some embodiments of the disclosed methods, uses, pharmaceutical compositions, and kits, treatment with the IL-17 antibody or antigen-binding fragment thereof reduces the burden of corticosteroid therapy, reduces the cumulative dose of corticosteroid, induces or maintains GCA remission, or a combination thereof.

In some embodiments of the disclosed uses, methods and kits, the IL-17 antagonist is an IL-17 antibody or antigen-binding fragment thereof. In some embodiments of the disclosed uses, methods and kits, the IL-17 antibody or antigen-binding fragment thereof is selected from the group consisting of: a) an IL-17 antibody or antigen-binding fragment thereof that binds to an epitope of IL-17 comprising Leu74, Tyr85, His86, Met87, Asn88, Val124, Thr125, Pro126, Ile127, Val128, His129; b) an IL-17 antibody or antigen-binding fragment thereof that binds to an epitope of IL-17 comprising Tyr43, Tyr44, Arg46, Ala79, Asp80; c) an IL-17 antibody or antigen-binding fragment thereof that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain; d) an IL-17 antibody or antigen-binding fragment thereof that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM (e.g., about 200 pM), and wherein the IL-17 antibody or antigen-binding fragment thereof has an in vivo half-life of about 23 to about 35 days (e.g., about 27 days); and e) an IL-17 antibody or antigen-binding fragment thereof comprising: i) an immunoglobulin heavy chain variable domain (V_{H}) comprising the amino acid sequence set forth as SEQ ID NO:8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO:1, SEQ ID NO: 2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO: 11, SEQ ID NO: 12 and SEQ ID NO: 13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO:14; x) an immunoglobulin heavy chain comprising the amino acid sequence set forth as SEQ ID NO:15; or xi) an immunoglobulin light chain comprising the amino acid sequence set forth as SEQ ID NO: 14 and an immunoglobulin heavy chain comprising the amino acid sequence set forth as SEQ ID NO: 15.

In some embodiments of the disclosed uses, methods and kits, the IL-17 antibody or antigen-binding fragment thereof is secukinumab (AIN457), a high-affinity recombinant, fully human monoclonal anti-human interleukin-17A antibody of the IgG1/κ-class.

Also disclosed herein are methods, uses, pharmaceutical compositions, and kits, for treating a patient having active GCA, comprising administering to the patient about 300 mg of secukinumab by subcutaneous injection at weeks 0, 1, 2, 3, and 4, and then every four weeks thereafter, for a total treatment duration of at least 26 weeks. Also disclosed herein are methods, uses, pharmaceutical compositions, and kits, for treating a patient having active GCA, comprising administering to the patient about 150 mg of secukinumab by subcutaneous injection at weeks 0, 1, 2, 3, and 4, and then every four weeks thereafter, for a total treatment duration of at least 26 weeks.

In some embodiments of the methods, uses, pharmaceutical compositions, and kits, the patient is naive to biological therapy, e.g., naive to treatment with a biological inhibitor of TNF-alpha, a biological IL-17 antagonist, a biological IL-6 inhibitor, or a combination thereof, prior to undergoing a treatment described herein. In some embodiments of the methods, uses, pharmaceutical compositions, and kits, the patient has active symptoms and/or signs of GCA and the elevated inflammatory markers CRP≥10 mg/L or ESR≥30 mm/hr due to GCA within 6 or 8 weeks of the first treatment with IL-17 therapy. If inflammatory markers showing active disease are not available (especially given that patients will have been treated with corticosteroids), disease activity can be confirmed by temporal artery biopsy (TAB) or diagnostic imaging (e.g. MRA, ultrasound). In some embodiments of the methods, uses, pharmaceutical compositions, and kits, the patient has new-onset or relapsing GCA. In some embodiments, new-onset GCA is defined as GCA that has recently been diagnosed, e.g., within 6 weeks. In some embodiments, relapsing GCA includes patients having been diagnosed for GCA>6 weeks before onset of a treatment described herein, and the patient has experienced a recurrence of active disease, e.g., following the institution of an appropriate non-anti-IL-17 treatment. For example, the relapse can occur following the institution of a corticosteroid treatment. In some embodiments of the methods, uses, pharmaceutical compositions, and kits, the patient is treated with vitamin D (1000 I.U. per day) and/or calcium supplements. In some embodiments, the patient has one or more cranial symptoms of GCA (new-onset localized headache, scalp or temporal artery tenderness, ischemia-related vision loss, or otherwise unexplained mouth or jaw pain upon mastication). In some embodiments, the treatment with anti-IL-17 reduces, alleviates, or eliminates one or more cranial symptoms of GCA (e.g., by at least 20%, or by at least 20% more compared to a standard course of corticosteroids).

### BRIEF DESCRIPTION OF THE FIGURE

FIG. 1 provides the giant cell arteritis clinical study design for Example 1. The study consists of 6-week (maximum duration) screening period, a 52-week treatment period and a 8-week safety follow-up period. Patients who do not achieve remission by Week 12, experience a flare after remission or cannot adhere (as described below) to the prednisolone taper regimen will enter “escape”. Upon entering “escape”, patients will receive prednisolone at a dose determined by the physician's clinical judgment and continue to receive secukinumab or placebo in a blinded manner. Patients in “escape” should continue to attend all subsequent scheduled visit assessments.

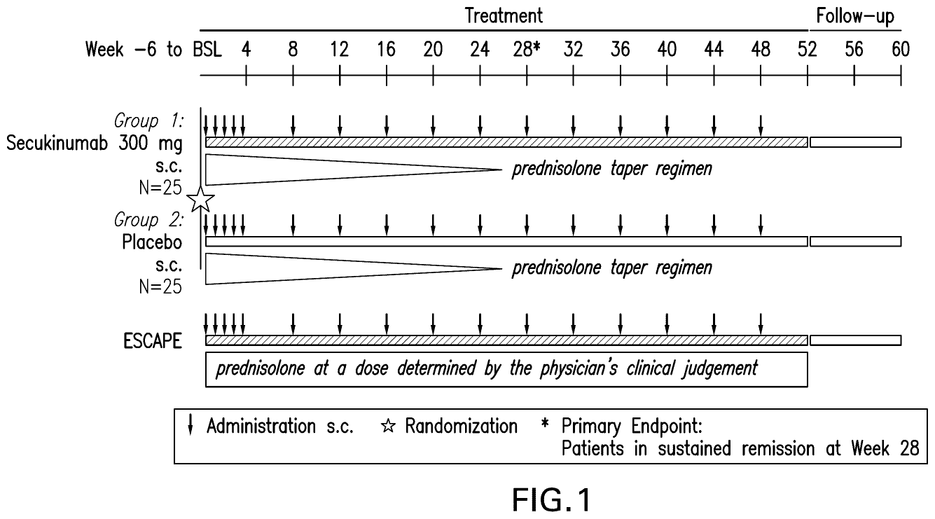

FIG. 2 Shows patient disposition after completion of the study described in Example 1.

FIG. 3 Shows baseline demographics and disease characteristics for the study described in Example 1.

FIG. 4 Shows the proportion of patients meeting the primary endpoint in Example 1.

FIG. 5 Shows the proportion of patients meeting the secondary endpoint in Example 1.

FIG. 6 Shows time to flare after baseline up to week 52 in Example 1.

FIG. 7 Shows the total cumulative dose of administered corticosteroid prednisolone in patients treated in Example 1.

FIG. 8 provides the giant cell arteritis clinical study design for Example 2. The study will consist of 6-week screening period, a 56-week treatment period (Treatment Period 1), a second treatment period of 52 weeks (Treatment Period 2) and an 8-week safety Follow-Up Period.

### DETAILED DESCRIPTION OF THE DISCLOSURE

As used herein, the phrase “failed to respond to” is used to mean that a patient's symptoms were not abrogated, treated, reduced, etc. in response to a particular GCA treatment. In some embodiments, the GCA patient failed to respond to a prior GCA treatment, e.g., treatment with a corticosteroid such as prednisone, prednisolone, or methylprednisolone, an IL-6 inhibitor, a TNF-alpha inhibitor, or combinations thereof.

As used herein, the phrase “had an inadequate response to” is used to mean that a patient's symptoms were not sufficiently abrogated, treated, reduced, etc. in response to a particular GCA treatment. In some embodiments, the GCA patient had an inadequate response to a prior GCA treatment, e.g., treatment with a corticosteroid such as prednisone, prednisolone, or methylprednisolone, an IL-6 inhibitor, a TNF-alpha inhibitor, or combinations thereof.

As used herein, the phrase “intolerant to” is used to mean that a patient experienced an adverse response to a particular GCA treatment. In some embodiments, the GCA patient was intolerant to a prior GCA treatment, e.g., treatment with a corticosteroid such as prednisone, prednisolone, or methylprednisolone, an IL-6 inhibitor, a TNF-alpha inhibitor, or combinations thereof.

As used herein, “fixed dose” refers to a flat dose, i.e., a dose that is not modified based on a patient's characteristics. Thus, a fixed dose differs from, e.g., a body-surface area-based dose or a weight-based dose (typically given as mg/kg). In preferred embodiments, the doses employed in the disclosed methods, uses, indications, kits, etc. are fixed doses. In most preferred embodiments, the patient is administered fixed doses of the IL-17 antibody, e.g., a fixed dose of secukinumab, e.g., a fixed dose of about 75 mg, about 150 mg, or about 300 mg of secukinumab.

As used herein, IL-17 refers to interleukin-17A (IL-17A).

As used herein, IL-17AF refers to the heterodimer consisting of a monomer of IL-17A and IL-17F.

The term “comprising” encompasses “including” as well as “consisting,” e.g., a composition “comprising” X may consist exclusively of X or may include something additional, e.g., X+Y.

As used herein, the phrase “TNF-alpha antagonist” refers to small molecules and biological molecules capable of inhibiting, reducing and/or blocking TNF-alpha signal, transduction, and/or activity. Examples of TNF-alpha antagonists include Enbrel® (etanercept), Humira® (adalimumab), Remicade® (infliximab) and Simponi® (golimumab).

Unless otherwise specifically stated or clear from context, as used herein, the term “about” in relation to a numerical value is understood as being within the normal tolerance in the art, e.g., within two standard deviations of the mean. Thus, “about” can be within +/−10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes naturally-occurring and whole antibodies. A naturally-occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Exemplary antibodies include secukinumab (Table 1) and ixekizumab (U.S. Pat. No. 7,838,638).

The term “antigen-binding fragment” of an antibody, as used herein, refers to fragments of an antibody that retain the ability to specifically bind to an antigen (e.g., IL-17). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F (ab) 2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen-binding sites include the CDRs of secukinumab as set forth in SEQ ID NOs: 1-6 and 11-13 (Table 1), preferably the heavy chain CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using techniques known to those of skill in the art.

An “isolated antibody”, as used herein, refers to an antibody that is substantially free of other antibodies having different antigenic specificities (e.g., an isolated antibody that specifically binds IL-17 is substantially free of antibodies that specifically bind antigens other than IL-17). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed processes and compositions, the IL-17 antibody is a human antibody, an isolated antibody, and/or a monoclonal antibody.

The term “IL-17” refers to IL-17A, formerly known as CTLA8, and includes wild-type IL-17A from various species (e.g., human, mouse, and monkey), polymorphic variants of IL-17A, and functional equivalents of IL-17A. Functional equivalents of IL-17A according to the present disclosure preferably have at least about 65%, 75%, 85%, 95%, 96%, 97%, 98%, or even 99% overall sequence identity with a wild-type IL-17A (e.g., human IL-17A), and substantially retain the ability to induce IL-6 production by human dermal fibroblasts.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system. In some embodiments, the IL-17 antibody or antigen-binding fragment thereof, e.g., secukinumab, binds human IL-17 with a K_{D} of about 1-250 pM, preferably about 100-200 pM (e.g., about 200 pM).

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IL-17 of various species are known in the art, including for example, ELISAs, western blots and RIAs. The binding kinetics (e.g., binding affinity) of the antibodies also can be assessed by assays known in the art, such as by Biacore® analysis.

An antibody that “inhibits” one or more of these IL-17 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IL-17 activity affects a statistically significant decrease, e.g., by at least about 10% of the measured parameter, by at least 50%, 80% or 90%, and in certain embodiments of the disclosed methods and compositions, the IL-17 antibody used may inhibit greater than 95%, 98% or 99% of IL-17 functional activity.

“Inhibit IL-6” as used herein refers to the ability of an IL-17 antibody or antigen-binding fragment thereof (e.g., secukinumab) to decrease IL-6 production from primary human dermal fibroblasts. The production of IL-6 in primary human (dermal) fibroblasts is dependent on IL-17 (Hwang et al., (2004) Arthritis Res Ther; 6: R120-128). In short, human dermal fibroblasts are stimulated with recombinant IL-17 in the presence of various concentrations of an IL-17 binding molecule or human IL-17 receptor with Fc part. The chimeric anti-CD25 antibody Simulect® (basiliximab) may be conveniently used as a negative control. Supernatant is taken after 16 h stimulation and assayed for IL-6 by ELISA. An IL-17 antibody or antigen-binding fragment thereof, e.g., secukinumab, typically has an IC₅₀ for inhibition of IL-6 production (in the presence 1 nM human IL-17) of about 50 nM or less (e.g., from about 0.01 to about 50 nM) when tested as above, i.e., said inhibitory activity being measured on IL-6 production induced by hu-IL-17 in human dermal fibroblasts. In some embodiments of the disclosed methods and compositions, IL-17 antibodies or antigen-binding fragments thereof, e.g., secukinumab, and functional derivatives thereof have an IC₅₀ for inhibition of IL-6 production as defined above of about 20 nM or less, more preferably of about 10 nM or less, more preferably of about 5 nM or less, more preferably of about 2 nM or less, more preferably of about 1 nM or less.

The term “derivative”, unless otherwise indicated, is used to define amino acid sequence variants, and covalent modifications (e.g., pegylation, deamidation, hydroxylation, phosphorylation, methylation, etc.) of an IL-17 antibody or antigen-binding fragment thereof, e.g., secukinumab, according to the present disclosure, e.g., of a specified sequence (e.g., a variable domain). A “functional derivative” includes a molecule having a qualitative biological activity in common with the disclosed IL-17 antibodies. A functional derivative includes fragments and peptide analogs of an IL-17 antibody as disclosed herein. Fragments comprise regions within the sequence of a polypeptide according to the present disclosure, e.g., of a specified sequence. Functional derivatives of the IL-17 antibodies disclosed herein (e.g., functional derivatives of secukinumab) preferably comprise V_{H} and/or V_{L} domains that have at least about 65%, 75%, 85%, 95%, 96%, 97%, 98%, or even 99% overall sequence identity with the V_{H} and/or V_{L} sequences of the IL-17 antibodies and antigen-binding fragments thereof disclosed herein, and substantially retain the ability to bind human IL-17 or, e.g., inhibit IL-6 production of IL-17 induced human dermal fibroblasts.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IL-17 antibody (e.g., a derivative of secukinumab, e.g., a secukinumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are known. The percent identity can be determined by standard alignment algorithms, for example, the Basic Local Alignment Search Tool (BLAST) described by Altshul et al. ((1990) J. Mol. Biol., 215:403 410); the algorithm of Needleman et al. ((1970) J. Mol. Biol., 48:444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure. Amino acid sequence variants of an antibody according to the present disclosure, e.g., of a specified sequence, still have the ability to bind the human IL-17 or, e.g., inhibit IL-6 production of IL-17 induced human dermal fibroblasts. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an IL-17 binding molecule or another agent, is used to refer to delivery of that compound to a patient by any route.

As used herein, a “therapeutically effective amount” refers to an amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., an IL-17 antagonist, e.g., secukinumab) administered alone, the term refers to that ingredient alone. When applied to a combination, the term refers to combined amounts of the active ingredients that result in the therapeutic effect, whether administered in combination, serially or simultaneously.

The term “treatment” or “treat” is herein defined as the application or administration of an IL-17 antibody according to the disclosure, for example, secukinumab or ixekizumab, or a pharmaceutical composition comprising said anti-IL-17 antibody, to a subject or to an isolated tissue or cell line from a subject, where the subject has a particular disease (e.g., GCA), a symptom associated with the disease (e.g., GCA), or a predisposition towards development of the disease (e.g., GCA) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse, or maintanence of remission.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating, and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology.

### IL-17 Antagonists

The various disclosed processes, kits, uses and methods utilize an IL-17 antagonist. IL-17 antagonists are capable of blocking, reducing and/or inhibiting IL-17 signal, activity and/or transduction. Examples of IL-17 antagonists include e.g., IL-17 binding molecules (e.g., soluble IL-17 receptors, IL-17 antibodies or antigen-binding fragments thereof, e.g., secukinumab and ixekizumab) and IL-17 receptor binding molecules (e.g., IL-17 receptor antibodies or antigen-binding fragments thereof, e.g., broadalumab). In some embodiments, the IL-17 antagonist is an IL-17 binding molecule, preferably an IL-17 antibody or antigen-binding fragment thereof. IL-17 antibodies and antigen-binding fragment thereof as used herein can be fully-human, CDR-grafted, or chimeric. It is preferable that the constant region domains of an antibody or antigen-binding fragment thereof for use in the disclosed methods, uses, kits, etc. preferably comprise suitable human constant region domains, for instance as described in “Sequences of Proteins of Immunological Interest”, Kabat E. A. et al, US Department of Health and Human Services, Public Health Service, National Institute of Health.

Particularly preferred IL-17 antibodies or antigen-binding fragments thereof used in the disclosed methods are human antibodies, especially secukinumab as described in Examples 1 and 2 of WO 2006/013107, which is incorporated by reference herein in its entirety. Secukinumab is a recombinant high-affinity, fully human monoclonal anti-human interleukin-17A (IL-17A, IL-17) antibody of the IgG₁/kappa isotype. Secukinumab has a high affinity for IL-17, i.e., a K_{D} of about 100-200 pM (e.g., about 200 pM), an IC₅₀ for in vitro neutralization of the biological activity of about 0.67 nM human IL-17A of about 0.4 nM, and a half-life of about 4 weeks.

For ease of reference, the amino acid sequences of the hypervariable regions of the secukinumab monoclonal antibody, based on the Kabat definition and as determined by the X-ray analysis and using the approach of Chothia and coworkers, is provided in Table 1, below.

TABLE 1
Amino acid sequences of the hypervariable regions
of secukinumab. The DNA encoding the V_{L} of
secukinumab is set forth in SEQ ID NO: 9.
The DNA encoding the V_{H} of secukinumab is
set forth in SEQ ID NO: 7.
Light-Chain

| | | |
|---|---|---|
| CDR1′ | Kabat | R-A-S-Q-S-V-S-S-S-Y-L-A |
| | | (SEQ ID NO: 4) |
| | Chothia | R-A-S-Q-S-V-S-S-S-Y-L-A |
| | | (SEQ ID NO: 4) |
| CDR2′ | Kabat | G-A-S-S-R-A-T |
| | | (SEQ ID NO: 5) |
| | Chothia | G-A-S-S-R-A-T |
| | | (SEQ ID NO: 5) |
| CDR3′ | Kabat | Q-Q-Y-G-S-S-P-C-T |
| | | (SEQ ID NO: 6) |
| | Chothia | Q-Q-Y-G-S-S-P-C-T |
| | | (SEQ ID NO: 6) |

Heavy-Chain

| | | |
|---|---|---|
| CDR1 | Kabat | N-Y-W-M-N |
| | | (SEQ ID NO: 1) |
| CDR1-x | Chothia | G-F-T-F-S-N-Y-W-M-N |
| | | (SEQ ID NO: 11) |
| CDR2 | Kabat | A-I-N-Q-D-G-S-E-K-Y-Y-V-G-S- |
| | | V-K-G (SEQ ID NO: 2) |
| CDR2-x | Chothia | A-I-N-Q-D-G-S-E-K-Y-Y |
| | | (SEQ ID NO: 12) |
| CDR3 | Kabat | D-Y-Y-D-I-L-T-D-Y-Y-I-H- |
| | | Y-W-Y-F-D-L (SEQ ID NO: 3) |
| CDR3-x | Chothia | C-V-R-D-Y-Y-D-I-L-T-D-Y-Y-I- |
| | | H-Y-W-Y-F-D-L-W-G |
| | | (SEQ ID NO: 13) |

In one embodiment, the IL-17 antibody or antigen-binding fragment thereof comprises at least one immunoglobulin heavy chain variable domain (V_{H}) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the IL-17 antibody or antigen-binding fragment thereof comprises at least one immunoglobulin light chain variable domain (V_{L′}) comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6. In one embodiment, the IL-17 antibody or antigen-binding fragment thereof comprises at least one immunoglobulin heavy chain variable domain (V_{H}) comprising hypervariable regions CDR1-x, CDR2-x and CDR3-x, said CDR1-x having the amino acid sequence SEQ ID NO: 11, said CDR2-x having the amino acid sequence SEQ ID NO:12, and said CDR3-x having the amino acid sequence SEQ ID NO:13.

In one embodiment, the IL-17 antibody or antigen-binding fragment thereof comprises at least one immunoglobulin V_{H} domain and at least one immunoglobulin V_{L} domain, wherein: a) the immunoglobulin V_{H} domain comprises (e.g., in sequence): i) hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO: 3; or ii) hypervariable regions CDR1-x, CDR2-x and CDR3-x, said CDR1-x having the amino acid sequence SEQ ID NO: 11, said CDR2-x having the amino acid sequence SEQ ID NO: 12, and said CDR3-x having the amino acid sequence SEQ ID NO:13; and b) the immunoglobulin V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen-binding fragment thereof comprises: a) an immunoglobulin heavy chain variable domain (V_{H}) comprising the amino acid sequence set forth as SEQ ID NO:8; b) an immunoglobulin light chain variable domain (V_{L}) comprising the amino acid sequence set forth as SEQ ID NO:10; c) an immunoglobulin V_{H} domain comprising the amino acid sequence set forth as SEQ ID NO:8 and an immunoglobulin V_{L} domain comprising the amino acid sequence set forth as SEQ ID NO:10; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO:4, SEQ ID NO: 5 and SEQ ID NO:6; f) an immunoglobulin V_{H} domain comprising the hypervariable regions set forth as SEQ ID NO:11, SEQ ID NO:12 and SEQ ID NO:13; g) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or h) an immunoglobulin V_{H} domain comprising the hypervariable regions set forth as SEQ ID NO:11, SEQ ID NO:12 and SEQ ID NO: 13 and an immunoglobulin V_{L} domain comprising the hypervariable regions set forth as SEQ ID NO: 4, SEQ ID NO:5 and SEQ ID NO:6.

In some embodiments, the IL-17 antibody or antigen-binding fragment thereof (e.g., secukinumab) comprises the three CDRs of SEQ ID NO:10. In other embodiments, the IL-17 antibody or antigen-binding fragment thereof comprises the three CDRs of SEQ ID NO:8. In other embodiments, the IL-17 antibody or antigen-binding fragment thereof comprises the three CDRs of SEQ ID NO: 10 and the three CDRs of SEQ ID NO:8. CDRs of SEQ ID NO:8 and SEQ ID NO: 10 may be found in Table 1. The free cysteine in the light chain (CysL97) may be seen in SEQ ID NO:6.

In some embodiments, IL-17 antibody or antigen-binding fragment thereof comprises the light chain of SEQ ID NO:14. In other embodiments, the IL-17 antibody or antigen-binding fragment thereof comprises the heavy chain of SEQ ID NO: 15 (with or without the C-terminal lysine). In other embodiments, the IL-17 antibody or antigen-binding fragment thereof comprises the light chain of SEQ ID NO: 14 and the heavy domain of SEQ ID NO:15. In some embodiments, the IL-17 antibody or antigen-binding fragment thereof comprises the three CDRs of SEQ ID NO: 14. In other embodiments, IL-17 antibody or antigen-binding fragment thereof comprises the three CDRs of SEQ ID NO:15. In other embodiments, the IL-17 antibody or antigen-binding fragment thereof comprises the three CDRs of SEQ ID NO: 14 and the three CDRs of SEQ ID NO: 15. CDRs of SEQ ID NO: 14 and SEQ ID NO: 15 may be found in Table 1.

Hypervariable regions may be associated with any kind of framework regions, though preferably are of human origin. Suitable framework regions are described in Kabat E. A. et al, ibid. The preferred heavy chain framework is a human heavy chain framework, for instance that of the secukinumab antibody. It consists in sequence, e.g. of FR1 (amino acid 1 to 30 of SEQ ID NO:8), FR2 (amino acid 36 to 49 of SEQ ID NO:8), FR3 (amino acid 67 to 98 of SEQ ID NO:8) and FR4 (amino acid 117 to 127 of SEQ ID NO:8) regions. Taking into consideration the determined hypervariable regions of secukinumab by X-ray analysis, another preferred heavy chain framework consists in sequence of FR1-x (amino acid 1 to 25 of SEQ ID NO:8), FR2-x (amino acid 36 to 49 of SEQ ID NO:8), FR3-x (amino acid 61 to 95 of SEQ ID NO:8) and FR4 (amino acid 119 to 127 of SEQ ID NO:8) regions. In a similar manner, the light chain framework consists, in sequence, of FR1′ (amino acid 1 to 23 of SEQ ID NO:10), FR2′ (amino acid 36 to 50 of SEQ ID NO: 10), FR3′ (amino acid 58 to 89 of SEQ ID NO:10) and FR4′ (amino acid 99 to 109 of SEQ ID NO: 10) regions.

In one embodiment, the IL-17 antibody or antigen-binding fragment thereof (e.g., secukinumab) is selected from a human IL-17 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO: 1, said CDR2 having the amino acid sequence SEQ ID NO:2, and said CDR3 having the amino acid sequence SEQ ID NO:3; and b) an immunoglobulin light chain or fragment thereof which comprises a variable domain comprising, in sequence, the hypervariable regions CDR1′, CDR2′, and CDR3′ and the constant part or fragment thereof of a human light chain, said CDR1′ having the amino acid sequence SEQ ID NO:4, said CDR2′ having the amino acid sequence SEQ ID NO: 5, and said CDR3′ having the amino acid sequence SEQ ID NO:6.

In one embodiment, the IL-17 antibody or antigen-binding fragment thereof is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO: 3; and b) a second domain comprising, in sequence, the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO: 6; and c) a peptide linker which is bound either to the N-terminal extremity of the first domain and to the C-terminal extremity of the second domain or to the C-terminal extremity of the first domain and to the N-terminal extremity of the second domain.

Alternatively, an IL-17 antibody or antigen-binding fragment thereof as used in the disclosed methods may comprise a derivative of the IL-17 antibodies set forth herein by sequence (e.g., a pegylated version of secukinumab). Alternatively, the V_{H} or V_{L} domain of an IL-17 antibody or antigen-binding fragment thereof used in the disclosed methods may have V_{H} or V_{L} domains that are substantially identical to the V_{H} or V_{L} domains set forth in SEQ ID NO:8 and 10. A human IL-17 antibody disclosed herein may comprise a heavy chain that is substantially identical to that set forth as SEQ ID NO:15 and/or a light chain that is substantially identical to that set forth as SEQ ID NO:14. A human IL-17 antibody disclosed herein may comprise a heavy chain that comprises SEQ ID NO: 15 and a light chain that comprises SEQ ID NO: 14. A human IL-17 antibody disclosed herein may comprise: a) one heavy chain, comprising a variable domain having an amino acid sequence substantially identical to that shown in SEQ ID NO:8 and the constant part of a human heavy chain; and b) one light chain, comprising a variable domain having an amino acid sequence substantially identical to that shown in SEQ ID NO: 10 and the constant part of a human light chain.

Alternatively, an IL-17 antibody or antigen-binding fragment thereof used in the disclosed methods may be an amino acid sequence variant of the reference IL-17 antibodies set forth herein, as long as it contains CysL97. The disclosure also includes IL-17 antibodies or antigen-binding fragments thereof (e.g., secukinumab) in which one or more of the amino acid residues of the V_{H} or V_{L} domain of secukinumab (but not CysL97), typically only a few (e.g., 1-10), are changed; for instance by mutation, e.g., site directed mutagenesis of the corresponding DNA sequences. In all such cases of derivative and variants, the IL-17 antibody or antigen-binding fragment thereof is capable of inhibiting the activity of about 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50%, said inhibitory activity being measured on IL-6 production induced by hu-IL-17 in human dermal fibroblasts as described in Example 1 of WO 2006/013107.

In some embodiments, the IL-17 antibodies or antigen-binding fragments thereof, e.g., secukinumab, bind to an epitope of mature human IL-17 comprising Leu74, Tyr85, His86, Met87, Asn88, Val124, Thr125, Pro126, Ile127, Val128, His129. In some embodiments, the IL-17 antibody, e.g., secukinumab, binds to an epitope of mature human IL-17 comprising Tyr43, Tyr44, Arg46, Ala79, Asp80. In some embodiments, the IL-17 antibody, e.g., secukinumab, binds to an epitope of an IL-17 homodimer having two mature human IL-17 chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain. The residue numbering scheme used to define IL-17 epitopes herein is based on residue one being the first amino acid of the mature protein (i.e., IL-17A lacking the 23 amino acid N-terminal signal peptide and beginning with Glycine). The sequence for immature IL-17A is set forth in the Swiss-Prot entry Q16552.

In some embodiments, the IL-17 antibody has a K_{D} of about 100-200 pM. In some embodiments, the IL-17 antibody has an IC₅₀ of about 0.4 nM for in vitro neutralization of the biological activity of about 0.67 nM human IL-17A. In some embodiments, the absolute bioavailability of subcutaneously (SC) administered IL-17 antibody has a range of about 60-about 80%, e.g., about 73%, about 76%. In some embodiments, the IL-17 antibody, such as secukinumab, has an elimination half-life of about 4 weeks (e.g., about 23 to about 35 days, about 23 to about 30 days, e.g., about 30 days). In some embodiments, the IL-17 antibody (such as secukinumab) has a Tₘₐₓ of about 7-8 days.

Other preferred IL-17 antagonists for use in the disclosed methods, kits and regimens include KHK4872 (Kyowa Hakko Kirin), ABT-122 (Abbvie), BCD-085 (JCS Biopharm), vidofludimus (4SC-101), NI-1401 (RG7624; MCAF5352A-NovImmune), ANB004 (AnaptysBio Inc.), E-036041 (Ensemble Therapeutics Corp.), Qβ-IL-17 (virus-like particle-based vaccine), PRS-190 (Pieris AG) bimekizumab (UCB 4940—UCB), ALX-0761, CNTO 6785 (Janssen Pharmaceuticals), LY3074828 (Eli Lilly), LY3114062 (Eli Lilly), SCH-900117, MSB0010841 (ALX-0761—Merck), ABT-122, COVA322 (Covagen), ixekizumab and brodalumab, as well as those set forth in U.S. Pat. Nos. 9,193,788; 8,057,794; 7,767,206; 8,003,099; 8,110,191; and 7,838,638 and US Published Patent Application Nos: 20120034656 and 20110027290, which are incorporated by reference herein as to their disclosure of the sequence and/or structure of an IL-17 antagonist.

In some embodiments of the disclosed uses, methods and kits, the IL-17 antibody or antigen-binding fragment thereof is selected from the group consisting of: a) an IL-17 antibody or antigen-binding fragment thereof that binds to an epitope of IL-17 between residues Arg 55 and Trp 67; b) an IL-17 antibody or antigen-binding fragment thereof that binds to an epitope of IL-17 comprising Arg 55, Glu 57, and Trp 67; c) an IL-17 antibody or antigen-binding fragment thereof that binds to an epitope of IL-17 comprising: Arg 55, Glu 57, Trp 67, Tyr 62, and Arg 101; d) an IL-17 antibody or antigen-binding fragment thereof that binds to an epitope of IL-17 comprising Arg 55, Glu 57, Trp 67, Tyr 62, Arg 101, Pro 59, Ser 64, and Val 65; e) an IL-17 antibody or antigen-binding fragment thereof that binds to an epitope of IL-17 comprising Arg 55, Glu 57, Trp 67, Tyr 62, Arg 101, Pro 59, Ser 64, Val 65, Val 22*, Leu 26, Asp 58, Glu 60, Pro 63, Pro 107, Phe 110, and Lys 114*, where amino acids marked with (*) designate a residue contributed by the second IL-17 subunit of the IL-17A homodimer, wherein the IL-17 antibody or antigen-binding fragment thereof has a K_{D} for human IL-17 of about 1-10 pM (e.g., about 6 pM), and wherein the IL-17 antibody or antigen-binding fragment thereof has an in vivo half-life of about 14-23 days, e.g., about 20 days; and f) an IL-17 antibody or antigen-binding fragment thereof comprising: i) an immunoglobulin heavy chain variable domain (V_{H}) comprising the amino acid sequence set forth as SEQ ID NO:30; ii) an immunoglobulin light chain variable domain (V_{L}) comprising the amino acid sequence set forth as SEQ ID NO:22; iii) an immunoglobulin V_{H} domain comprising the amino acid sequence set forth as SEQ ID NO:30 and an immunoglobulin V_{L} domain comprising the amino acid sequence set forth as SEQ ID NO:22; iv) an immunoglobulin V_{H} domain comprising the hypervariable regions set forth as SEQ ID NO: 24, SEQ ID NO:26, and SEQ ID NO:28; v) an immunoglobulin V_{L} domain comprising the hypervariable regions set forth as SEQ ID NO: 16, SEQ ID NO: 18 and SEQ ID NO:20; vi) an immunoglobulin V_{H} domain comprising the hypervariable regions set forth as SEQ ID NO:25, SEQ ID NO:27 and SEQ ID NO:29; vii) an immunoglobulin V_{L} domain comprising the hypervariable regions set forth as SEQ ID NO:17, SEQ ID NO:19 and SEQ ID NO:21; viii) an immunoglobulin V_{H} domain comprising the hypervariable regions set forth as SEQ ID NO:24, SEQ ID NO:26, and SEQ ID NO:28 and an immunoglobulin V_{L} domain comprising the hypervariable regions set forth as SEQ ID NO: 16, SEQ ID NO: 18 and SEQ ID NO:20; ix) an immunoglobulin V_{H} domain comprising the hypervariable regions set forth as SEQ ID NO:25, SEQ ID NO:27, and SEQ ID NO:29 and an immunoglobulin V_{L} domain comprising the hypervariable regions set forth as SEQ ID NO:17, SEQ ID NO:19 and SEQ ID NO:21; x) a light chain comprising SEQ ID NO:23; xi) a heavy chain comprising SEQ ID NO:31; or xii) a light chain comprising SEQ ID NO:23 and a heavy chain comprising SEQ ID NO:31.

In some embodiments of the disclosed uses, methods and kits, the IL-17 antibody or antigen-binding fragment thereof is CJM112, a high-affinity recombinant, fully human monoclonal anti-human IL-17 antibody of the IgG₁/κ-class. CJM112 also binds to IL-17AF and antagonizes this cytokine (see, e.g., U.S. Pat. No. 9,193,788, which is incorporated by reference herein in its entirety).

IL-17AF antagonists may also find use in treating GCA by, e.g., inducing a reduction in vessel (e.g., large vessel or arterial) inflammation (vasculitis) in patients having GCA. These antagonists, which include antibodies cross-reactive with IL-17A and IL-17F, and bispecific anti-IL-17A/F antibodies, FynomAbs (e.g., COVA322), nanobodies (e.g., ALX-0761), etc. may be found in US Published Patent Application Nos. 20140314763, 2013/0195872, 20160326241, U.S. Pat. Nos. 8,496,936, 8,945,553, and published PCT Application No. WO/2016/070062.

### Methods of Treatment and Uses of IL-17 Antagonists for GCA

The disclosed IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof), may be used in vitro, ex vivo, or incorporated into pharmaceutical compositions and administered in vivo to treat GCA (e.g., human patients having GCA, such as active GCA).

In some embodiments, the patient has one or more or all inclusion criteria set forth in Example 1. In some embodiments, the patient has one or more or all inclusion criteria set forth in Example 2. In some embodiments, the patient does not have one or more or all exclusion criteria set forth in Example 1. In some embodiments, the patient does not have one or more or all exclusion criteria set forth in Example 2.

In some embodiments, the patient is biological-naïve. In some embodiments, the patient was previously treated with a biological for the GCA. In some embodiments, the patient was previously treated with a biological for a different disease but has not been treated with an alternative biological for the GCA. In some embodiments, the patient has inadquately responded to, or did not respond to, a previous biological treatment for GCA.

Response of patients to the treatments disclosed herein may be measured using patient reported outcomes (PROs), clinician reported outcomes, laboratory parameters, imaging techniques, etc. Clinician reported outcomes include, e.g., Physician's Global Assessment (PhGA) of disease activity (e.g., using a VAS score). Imaging techniques include, e.g., MRI (as described in the follow-up of the phase II tocilizumab tudy by Reichenbach et al (2018)); and ultrasound. PROs include, e.g., the Patient's global assessment (PGA) of disease activity (e.g., using a VAS score), FACIT-Fatigue score, SF36 score, and EQ-5D-5L score. Laboratory parameters include C reactive protein (CRP) levels and/or erythrocyte sedimentation rate (ESR).

EuroQuol 5D (EQ-5D-5L) (euroqol.org/eq-5d-instruments/eq-5d-5l-about/) is a widely used, self-administered questionnaire designed to assess health status in adults (Xu et al 2011, Mc Clure et al 2017). The purpose of the EQ-5D-5L in this study is to assess the general health status of the patients. The FACIT-Fatigue© is a 13-item questionnaire (Cella 1993 and Yellen 1997) that assesses self-reported fatigue and its impact upon daily activities and function. The SF-36 is a widely used and extensively studied instrument to measure health-related quality of life among healthy subjects and patients with acute and chronic conditions. It consists of eight subscales that can be scored individually: Physical Functioning, Role-Physical, Bodily Pain, General Health, Vitality, Social Functioning, Role-Emotional, and Mental Health (Ware 1993). Two overall summary scores, the Physical Component Summary (PCS) and the Mental Component Summary (MCS) also can be computed (Ware 1994).

A Visual Analogue Scale (VAS) is an instrument that measures a characteristic or attitude that is believed to range across a continuum of values. For example, the amount of pain that a patient feels ranges across a continuum from none to an extreme amount of pain. From the patient's perspective this spectrum appears continuous±their pain does not take discrete jumps, as a categorization of none, mild, moderate and severe would suggest. The VAS pain scale is commonly used as an outcome measurement to characterize pain intensity in clinical studies. It is usually presented as a 100-mm horizontal line on which the patient's pain intensity is represented by a point between the extremes of “no pain at all” and “worst pain imaginable” (or the like). Respondents are asked to place a line perpendicular to the VAS line at the point that best indicates their pain at the present time. The tool is scored in millimeters (although scores are often recorded in tenths of centimeters using a 10-point scale); commonly a score below 40 millimeters (or 4 if recorded in centimeters) is considered desirable for chronic pain management.

In some embodiments, the patient experiences at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% decrease in pain, decrease in inflammation, improvement in one or more PRO or physician reported outcomes, or laboratory or imaging parameters following 1, 2, 3, 4, 5, 6, 7, 8, 9, 10, 11 or 12 months (preferably 1, 2 or 3 months, more preferably 6, 12, or 24 months, or more) of treatment according to the claimed methods. In preferred embodiments, the patient experiences at least 20% decrease in pain, decrease in inflammation, following treatment according to the claimed methods.

In some embodiments, the patient experiences at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% improvement in VAS score, PGA score, FACIT-Fatique, SF-36, and/or EQ-5D-5L score following 1, 2, 3, 4, 5, 6, 7, 8, 9, 10, 11 or 12 months (preferably 1, 2 or 3 months) of treatment according to the claimed methods. In preferred embodiments, the patient experiences at least 20% decrease in pain, as determined by a VAS score, following treatment according to the claimed methods. In preferred embodiments, the patient experiences at least 20% improvement overall, as determined by a PGA score, following treatment according to the claimed methods.

The IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof), may be used as a pharmaceutical composition when combined with a pharmaceutically acceptable carrier. Such a composition may contain, in addition to an IL-17 antagonist, carriers, various diluents, fillers, salts, buffers, stabilizers, solubilizers, and other materials. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with the IL-17 binding molecules, or to minimize side effects caused by the IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof).

Pharmaceutical compositions for use in the disclosed methods may be manufactured in a known manner. In one embodiment, the pharmaceutical composition is provided in lyophilized form. For immediate administration it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinised blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise liquid or lyophilized formulation.

Antibodies, e.g., antibodies to IL-17, are typically formulated either in aqueous form ready for parenteral administration or as lyophilisates for reconstitution with a suitable diluent prior to administration. In some embodiments of the disclosed methods and uses, the IL-17 antagonist, e.g., IL-17 antibody, e.g., secukinumab, is formulated as a ready-to-use liquid pharmaceutical composition. Suitable lyophilisate formulations can be reconstituted in a small liquid volume (e.g., 2 ml or less) to allow subcutaneous administration and can provide solutions with low levels of antibody aggregation. The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are known. When a therapeutically effective amount of an IL-17 antagonist, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof) is administered by intravenous, cutaneous or subcutaneous injection, the IL-17 antagonist will be in the form of a pyrogen-free, parenterally acceptable solution. A pharmaceutical composition for intravenous, cutaneous, or subcutaneous injection may contain, in addition to the IL-17 antagonist, an isotonic vehicle such as sodium chloride, Ringer's solution, dextrose, dextrose and sodium chloride, lactated Ringer's solution, or other vehicle as known in the art.

In preferred embodiments of the disclosed methods, uses, kits, etc., the IL-17 binding molecule (e.g., IL-17 antibody or antigen-binding fragment thereof) or IL-17 receptor binding molecule (e.g., IL-17 antibody or antigen-binding fragment thereof) is secukinumab, which is provided in a stable liquid pharmaceutical formulation comprising about 25 mg/mL to about 150 mg/mL secukinumab, about 10 mM to about 30 mM histidine pH 5.8, about 200 mM to about 225 mM trehalose, about 0.02% polysorbate 80, and about 2.5 mM to about 20 mM methionine, wherein the liquid formulation is not reconstituted from a lyophilisate. One preferred pharmaceutical product for use in the disclosed methods, uses, kits, etc., comprises a stable liquid formulation having 150 mg/ml secukinumab in 20 mM histidine buffer, pH 5.8, 200 mM trehalose, 0.02% polysorbate 80 and 5 mM L-methionine, which is provided in a pre-filled syringe or an autoinjector (i.e., having 1 mL or 2 mL of the formulation).

The appropriate dosage will vary depending upon, for example, on the particular IL-17 antagonists employed, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof), the host, the mode of administration and the nature and severity of the condition being treated, and on the nature of prior treatments that the patient has undergone. Ultimately, the attending health care provider will decide the amount of the IL-17 antagonist with which to treat each individual patient. In some embodiments, the attending health care provider may administer low doses of the IL-17 antagonist and observe the patient's response. In other embodiments, the initial dose(s) of IL-17 antagonist administered to a patient are high, and then are titrated downward until signs of relapse occur. Larger doses of the IL-17 antagonist may be administered until the optimal therapeutic effect is obtained for the patient, and the dosage is not generally increased further.

In practicing some of the methods of treatment or uses of the present disclosure, a therapeutically effective amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) is administered to a patient, e.g., a mammal (e.g., a human). While it is understood that the disclosed methods provide for treatment of GCA patients using an IL-17 antagonist (e.g., secukinumab), this does not preclude that, if the patient is to be ultimately treated with an IL-17 antagonist, such IL-17 antagonist therapy is necessarily a monotherapy. Indeed, if a patient is selected for treatment with an IL-17 antagonist, then the IL-17 antagonist (e.g., secukinumab) may be administered in accordance with the methods of the disclosure either alone or in combination with other agents and therapies (e.g., other standard of care therapies for GCA, e.g., corticosteroids, IL-6 inhibitors, anti-TNF-alpha, methotrexate, etc.).

When co-administered with one or more additional GCA agents, an IL-17 antagonist may be administered either simultaneously with the other agent, or sequentially. If administered sequentially, the attending physician will decide on the appropriate sequence of administering the IL-17 antagonist in combination with other agents and the appropriate dosages for co-delivery. An IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) is conveniently administered parenterally, e.g., intravenously (e.g., into the antecubital or other peripheral vein), intramuscularly, or subcutaneously.

The duration of therapy using a pharmaceutical composition of the present disclosure will vary, depending on the severity of the disease being treated and the condition and personal response of each individual patient. The health care provider will decide on the appropriate duration of therapy and the timing of administration of the therapy, using the pharmaceutical composition of the present disclosure. As used herein, the phrase “total treatment duration” refers to the whole amount of time during which the patient is treated with the IL-17 antagonist, including the induction period (e.g., initial weekly dosing), if applicable. Thus, e.g., if the patient is administered the IL-17 antagonist weekly during week 0, 1, 2, 3, and 4, and then every 4 weeks for a total treatment duration of 2 months, then the patient is dosed during week 0, 1, 2, 3, 4, and 8. Similarly, e.g., if the patient is administered the IL-17 antagonist weekly during week 0, 1, 2, 3, and 4, and then every 4 weeks for a total treatment duration of 3 months, then the patient is dosed during week 0, 1, 2, 3, 4, 8, and 12.

Preferred total treatment duration is between 1-3 months, 3-6 months, 6-9 months, or 9-12 months. In some embodiments, the patient is treated for 3 months or less, e.g., 1, 2, or 3 months. In other embodiments, the patient is treated for up to 12 months, e.g., 1, 2, 3, 4, 5, 6, 7, 8, 9, 10, 11 or 12 months. Most preferred total treatment duration is 1, 2, or 3 months.

Preferred SC treatment regimens (including both induction and maintenance regimens) using secukinumab, which may be employed in treating GCA patients, are provided in PCT Application No. PCT/US2011/064307 and PCT/IB2014/063902, which, as applicable, are incorporated by reference herein in their entirety.

In some embodiments, a patient may be administered a single S.C. dose of about 150 mg-about 300 mg (e.g., about 150 mg, about 300 mg) of the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof).

In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient SC at about 150 mg-about 300 mg (e.g., about 150 mg, about 300 mg) weekly during weeks 0, 1, 2, 3 and 4.

In preferred embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient SC at a fixed dose of about 150 mg-about 300 mg (e.g., about 150 mg, about 300 mg) weekly during weeks 0, 1, 2, 3 and 4, and thereafter administered to the patient SC at about 150 mg-about 300 mg (e.g., about 150 mg, about 300 mg) every 4 weeks (monthly). In this manner, the patient is dosed SC with about 150 mg-about 300 mg (e.g., about 150 mg, about 300 mg) of the IL-17 antagonist (e.g., secukinumab) during weeks 0, 1, 2, 3, 4, 8, etc.

In other embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient without a loading regimen, e.g., the antagonist may be administered to the patient SC as a fixed dose of about 150 mg-about 300 mg (e.g., about 150 mg, about 300 mg) every 4 weeks (monthly). In this manner, the patient is dosed SC with about 150 mg-about 300 mg (e.g., about 150 mg, about 300 mg) of the IL-17 antagonist (e.g., secukinumab) during weeks 0, 4, 8, 12, etc. Ideally the patient, e.g., a patient having GCA, is administered the IL-17 antagonist (e.g., secukinumab) monthly (every 4 weeks), for a total of four doses (week 0, 4, 8, and 12).

In other preferred embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient bimonthly (every 2 weeks, every other week), every other month, quarterly (every three months), biyearly (every 6 months), or yearly.

Preferred S.C. doses (e.g., fixed doses) are about 150 mg-about 300 mg, preferably about 150 mg or about 300 mg. However, it will be understood that dose escalation may be required (e.g., during an induction and/or maintenance phase) for certain patients, e.g., patients that display inadequate response to treatment with the IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof). Thus, SC dosages of the IL-17 antagonists, e.g., secukinumab, may be greater than about 150 mg-about 300 mg, e.g., about 175 mg, about 200 mg, about 250 mg, about 350 mg, about 400 mg, about 450 mg, about 500 mg, about 600 mg, etc. It will also be understood that dose reduction may also be required (e.g., during the induction and/or maintenance phase) for certain patients, e.g., patients that display adverse events or an adverse response to treatment with the IL-17 antagonist (e.g., IL-17 antibody or antigen-binding fragment thereof, e.g., secukinumab). Thus, dosages of the IL-17 antagonist (e.g., IL-17 antibody or antigen-binding fragment thereof, e.g., secukinumab), may be less than about 150 mg to about 300 mg SC, e.g., about 75 mg, about 100 mg, about 125 mg, about 175 mg, about 200 mg, about 250 mg, about 275 mg, etc. In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient at an initial dose (or doses) of 150 mg delivered SC, and the dose is then escalated to about 300 mg if needed, as determined by a physician. In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient at an initial dose (or doses) of 300 mg delivered SC, and the dose is then escalated to about 450 mg if needed, as determined by a physician.

The timing of dosing is generally measured from the day of the first dose of drug (which is also known as “baseline”). However, health care providers often use different naming conventions to identify dosing schedules, as shown in Table 2.

TABLE 2
Common naming conventions for dosing regimens. Bolded
items refer to the naming convention used herein.

| Week | 0/1 | 1/2 | 2/3 | 3/4 | 4/5 | 5/6 | 6/7 | 7/8 | 8/9 | 9/10 | 10/11 | etc |
|---|---|---|---|---|---|---|---|---|---|---|---|---|
| 1^{st} | 0/1 | 7/8 | 14/15 | 21/22 | 28/29 | 35/36 | 42/43 | 49/50 | 56/57 | 63/64 | 70/71 | etc. |
| day | | | | | | | | | | | | |
| of | | | | | | | | | | | | |
| week | | | | | | | | | | | | |

Notably, week zero may be referred to as week one by some health care providers, while day zero may be referred to as day one by some health care providers. Thus, it is possible that different physicians will designate, e.g., a dose as being given during week 3/on ˜day 21, during week 3/on ˜day 22, during week 4/on ˜day 21, during week 4/on ˜day 22, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the IL-17 antibody, regardless of whether the physician refers to a particular week as “week 1” or “week 2”. In one dosing regimen, the antibody is administered monthly during week 0, 4, 8, 12, etc. In one dosing regimen, the antibody is administered during week 0, 1, 2, 3, 4, 8, 12, etc. Some providers may refer to this regimen as weekly for five weeks and then monthly (or every 4 weeks) thereafter, beginning during week 8, while others may refer to this regimen as weekly for four weeks and then monthly (or every 4 weeks) thereafter, beginning during week 4. It will be appreciated by a skilled artisan that administering a patient an injection at weeks 0, 1, 2 and 3, followed by once monthly (every 4 weeks) dosing starting at week 4 is the same as: 1) administering the patient an injection at weeks 0, 1, 2, 3, and 4, followed by monthly dosing starting at week 8; 2) administering the patient an injection at weeks 0, 1, 2, 3 and 4 followed by dosing every 4 weeks; and 3) administering the patient an injection at weeks 0, 1, 2, 3 and 4 followed by monthly administration.

Disclosed herein are methods of treating a patient having GCA, comprising administering a therapeutically effective amount of an IL-17 antibody or antigen-binding fragment thereof to a patient in need thereof, wherein the IL-17 antibody or antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

Additionally disclosed herein are therapeutically effective amounts of IL-17 antagonists (e.g., IL-17 antibody or antigen-binding fragment thereof, e.g., secukinumab) for use in treating a patient having GCA, wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in the manufacture of a medicament for treating a patient having GCA, wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

Additionally disclosed herein are IL-17 antagonists (e.g., IL-17 antibody or antigen-binding fragment thereof, e.g., secukinumab) for use in the manufacture of a medicament for treating a patient having GCA, wherein the medicament is formulated to comprise containers, each container having a sufficient amount of the IL-17 antagonist (e.g., IL-17 antibody or antigen-binding fragment thereof, e.g., secukinumab) to allow subcutaneous delivery of at least about 150 mg-about 300 mg (e.g., about 150 mg, about 300 mg) of the IL-17 antagonist (e.g., IL-17 antibody or antigen-binding fragment thereof, e.g., secukinumab) per unit dose, and further wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

As used herein, the phrase “container having a sufficient amount of the IL-17 antagonist to allow delivery of [a designated dose]” is used to mean that a given container (e.g., vial, pen, syringe) has disposed therein a volume of an IL-17 antagonist (e.g., as part of a pharmaceutical composition) that can be used to provide a desired dose. As an example, if a desired dose is 300 mg, then a clinician may use 2 ml from a container that contains an IL-17 antibody formulation with a concentration of 150 mg/ml, 1 ml from a container that contains an IL-17 antibody formulation with a concentration of 300 mg/ml, 0.5 ml from a container contains an IL-17 antibody formulation with a concentration of 600 mg/ml, etc. In each such case, these containers have a sufficient amount of the IL-17 antagonist to allow delivery of the desired 300 mg dose. In one embodiment, the container has disposed therein 1 ml of a formulation comprising 150 mg/ml secukinumab. In another embodiment, the container has disposed therein 2 ml of a formulation comprising 150 mg/ml secukinumab. Preferred formulations are liquid pharmaceutical compositions comprising about 25 mg/mL to about 150 mg/mL secukinumab, about 10 mM to about 30 mM histidine pH 5.8, about 200 mM to about 225 mM trehalose, about 0.02% polysorbate 80, and about 2.5 mM to about 20 mM methionine.

Additionally disclosed herein are IL-17 antagonists (e.g., IL-17 antibody or antigen-binding fragment thereof, e.g., secukinumab) for use in the manufacture of a medicament for treating a patient having GCA, wherein the medicament is formulated at a dosage to allow subcutaneous delivery of about 150 mg-about 300 mg (e.g., about 150 mg, about 300 mg) of the IL-17 antagonist (e.g., IL-17 antibody or antigen-binding fragment thereof, e.g., secukinumab) to the patient, and further wherein the IL-17 antagonist (e.g., IL-17 antibody or antigen-binding fragment thereof, e.g., secukinumab) binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

As used herein, the phrase “formulated at a dosage to allow [route of administration] delivery of [a designated dose]” is used to mean that a given pharmaceutical composition can be used to provide a desired dose of an IL-17 antagonist, e.g., an IL-17 antibody, e.g., secukinumab, via a designated route of administration (e.g., SC or IV). As an example, if a desired subcutaneous dose is 300 mg, then a clinician may use 2 ml of an IL-17 antibody formulation having a concentration of 150 mg/ml, 1 ml of an IL-17 antibody formulation having a concentration of 300 mg/ml, 0.5 ml of an IL-17 antibody formulation having a concentration of 600 mg/ml, etc. In each such case, these IL-17 antibody formulations are at a concentration high enough to allow subcutaneous delivery of the IL-17 antibody. Subcutaneous delivery typically requires delivery of volumes of ≤2 ml. In one embodiment, the patient is to be administered one 2 ml SC injection of a formulation containing 150 mg/ml secukinumab. In another embodiment, the patient is to be administered two 1 ml SC injections of a formulation containing 150 mg/ml secukinumab.

Disclosed herein are methods, uses, pharmaceutical compositions, and kits for treating GCA in a patient having GCA, comprising subcutaneously administering to a patient in need thereof about 150 mg-about 300 mg of an IL-17 antibody or antigen-binding fragment thereof, wherein the IL-17 antibody or antigen-binding fragment thereof binds to an epitope of a human IL-17 homodimer having two mature human IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

In some embodiments of the disclosed methods, uses, pharmaceutical compositions, and kits, the patient is administered the IL-17 antibody or antigen-binding fragment thereof only once.

In some embodiments of the disclosed methods, uses, pharmaceutical compositions, and kits, the patient is administered the IL-17 antibody or antigen-binding fragment thereof weekly.

In some embodiments of the disclosed methods, uses, pharmaceutical compositions, and kits, the patient is administered the IL-17 antibody or antigen-binding fragment thereof during week 0, 1, 2, 3, and 4.

In some embodiments of the disclosed methods, uses, pharmaceutical compositions, and kits, the patient is administered the IL-17 antibody or antigen-binding fragment thereof every 4 weeks.

In some embodiments of the disclosed methods, uses, pharmaceutical compositions, and kits, the patient is administered the IL-17 antibody or antigen-binding fragment thereof for a total treatment duration of at least two months.

In some embodiments of the disclosed methods, uses, pharmaceutical compositions, and kits, the patient is administered the IL-17 antibody or antigen-binding fragment thereof for a total treatment duration of at least four months.

In some embodiments of the disclosed methods, uses, pharmaceutical compositions, and kits, the patient is administered the IL-17 antibody or antigen-binding fragment thereof weekly during week 0, 1, 2, 3, and 4, and then every 4 weeks.

In some embodiments of the disclosed methods, uses, pharmaceutical compositions, and kits, the patient is administered the IL-17 antibody or antigen-binding fragment thereof during week 0, 1, 2, 3, 4, 8 and 12.

In some embodiments of the disclosed methods, uses, pharmaceutical compositions, and kits, the patient is administered the IL-17 antibody or antigen-binding fragment thereof weekly during week 0, 1, 2, 3, and 4, and then every 4 weeks thereafter, for a total treatment duration of at least three months.

In some embodiments of the disclosed methods, uses, pharmaceutical compositions, and kits, prior to treatment with the IL-17 antibody or antigen-binding fragment thereof, the patient failed to respond to, had an inadequate response to, or was intolerant to a prior GCA treatment selected from the group consisting of corticosteroid, anti-IL-6, anti-TNF-alpha, methotrexate, and combinations thereof.

In some embodiments of the disclosed methods, uses, pharmaceutical compositions, and kits, the patient has active GCA.

In some embodiments of the disclosed methods, uses, pharmaceutical compositions, and kits, the patient experiences at least a 20% improvement in one or more clinical, laboratory, or patient reported outcomes, or physician assessment, attributable to GCA following treatment with the IL-17 antibody or antigen-binding fragment thereof.

In some embodiments of the disclosed uses, methods, pharmaceutical compositions, and kits, the IL-17 antibody or antigen-binding fragment thereof comprises: i) an immunoglobulin heavy chain variable domain (V_{H}) comprising the amino acid sequence set forth as SEQ ID NO: 8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO:1, SEQ ID NO:2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO:11, SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO:11, SEQ ID NO:12 and SEQ ID NO: 13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO:14; x) an immunoglobulin heavy chain comprising the amino acid sequence set forth as SEQ ID NO: 15; or xi) an immunoglobulin light chain comprising the amino acid sequence set forth as SEQ ID NO:14 and an immunoglobulin heavy chain comprising the amino acid sequence set forth as SEQ ID NO: 15. In some embodiments of the disclosed uses, methods, and kits, the IL-17 antibody or antigen-binding fragment thereof is secukinumab.

Disclosed herein are also methods, uses, pharmaceutical compositions, and kits, for treating a patient having active GCA, comprising administering to the patient about 300 mg of secukinumab by subcutaneous injection at weeks 0, 1, 2, 3, and 4, and then every four weeks thereafter, for a total treatment duration of at least three months.

Disclosed herein are also methods, uses, pharmaceutical compositions, and kits, for treating a patient having active overuse GCA, comprising administering to the patient about 150 mg of secukinumab by subcutaneous injection at weeks 0, 1, 2, 3, and 4, and then every four weeks thereafter, for a total treatment duration of at least three months.

In some embodiments of the disclosed methods, uses, pharmaceutical compositions, and kits, prior to treatment with secukinumab, the patient was refractory to a prior GCA treatment selected from the group consisting of corticosteroid, anti-TNF-alpha, anti-IL-6, methotrexate, and combinations thereof.

### Kits

The disclosure also encompasses kits for treating a GCA patient. Such kits comprise a therapeutically effective amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) (e.g., in liquid or lyophilized form) or a pharmaceutical composition comprising a therapeutically effective amount of an IL-17 antagonist (described supra). Additionally, such kits may comprise means for administering the IL-17 antagonist (e.g., an autoinjector, a syringe and vial, a prefilled syringe, a prefilled pen) and instructions for use. These kits may contain additional therapeutic agents (described supra) for treating GCA, e.g., for delivery in combination with the enclosed IL-17 antagonist, e.g., IL-17 binding molecule, e.g., IL-17 antibody, e.g., secukinumab. Such kits may also comprise instructions for administration of the IL-17 antagonist (e.g., IL-17 antibody, e.g., secukinumab) to treat the GCA patient. Such instructions may provide the dose (e.g., about 150 mg-about 300 mg, e.g., about 150 mg, about 300 mg), route of administration (e.g., IV, SC, IM), regimen (e.g., weekly during week 0, 1, 2, 3, and 4; weekly during week 0, 1, 2, 3, and 4, and then every 4 weeks), and total treatment duration (e.g., 1, 2, 3, 4, 6, 8, 12 months, etc. [preferably 3-12 months, e.g., 3-6 months, e.g., 3 months]) for use with the enclosed IL-17 antagonist, e.g., IL-17 binding molecule, e.g., IL-17 antibody, e.g., secukinumab.

The phrase “means for administering” is used to indicate any available implement for systemically administering a drug to a patient, including, but not limited to, a pre-filled syringe, a vial and syringe, an injection pen, an autoinjector, an IV drip and bag, a pump, etc. With such items, a patient may self-administer the drug (i.e., administer the drug without the assistance of a physician) or a medical practitioner may administer the drug. In some embodiments, a total dose of 300 mg is to be delivered in a total volume of 2 ml having 150 mg/ml of the IL-17 antibody, e.g., secukinumab, which is disposed in a single PFS or autoinjector. In some embodiments, a total dose of 300 mg is to be delivered in a total volume of 2 ml which is disposed in two PFSs or autoinjectors, each PFS or autoinjector containing a volume of 1 ml having 150 mg/ml of the IL-17 antibody, e.g., secukinumab.

Disclosed herein are kits for use in treating a patient having GCA, comprising an IL-17 antagonist (e.g., IL-17 binding molecule, e.g., IL-17 antibody or antigen-binding fragment thereof, e.g., secukinumab). In some embodiments, the kit further comprises means for administering the IL-17 antagonist to the patient. In some embodiments, the kit further comprises instructions for administration of the IL-17 antagonist, wherein the instructions indicate that the IL-17 antagonist (e.g., IL-17 binding molecule, e.g., IL-17 antibody or antigen-binding fragment thereof, e.g., secukinumab) is to be administered to the patient subcutaneously (SC) at a dose of about 150 mg-about 300 mg (e.g., about 150 mg, or about 300 mg). In some embodiments, the instructions indicate that the IL-17 antagonist (e.g., IL-17 binding molecule, e.g., IL-17 antibody or antigen-binding fragment thereof, e.g., secukinumab) is to be administered to the patient weekly during weeks 0, 1, 2, 3, and 4. In some embodiments, the instructions indicate that the IL-17 antagonist (e.g., IL-17 binding molecule, e.g., IL-17 antibody or antigen-binding fragment thereof, e.g., secukinumab) is to be administered to the patient every 4 weeks (monthly) for a total treatment duration of at least 2 months, e.g., 2, 3, 4, 5, 6, 7, 8, 9, 10, 11 or 12 months (preferably 2 or 3 months). In some embodiments, the instructions indicate that the IL-17 antagonist (e.g., IL-17 binding molecule, e.g., IL-17 antibody or antigen-binding fragment thereof, e.g., secukinumab) is to be administered to the patient weekly during weeks 0, 1, 2, 3, and 4, and every 4 weeks (monthly) thereafter, for a total treatment duration of at least 3 months, i.e., 3, 4, 5, 6, 7, 8, 9, 10, 11 or 12 months (preferably 2 or 3 months). In some embodiments, the instructions will provide for dose escalation (e.g., from a dose of about 150 mg to a higher dose of about 300 mg, or from a dose of about 300 mg to a dose of about 450 mg), as needed, to be determined by a physician.

### General

In preferred embodiments of the disclosed methods, treatments, medicaments, regimens, uses and kits, the IL-17 antagonist is an IL-17 binding molecule. In preferred embodiments, the IL-17 binding molecule is an IL-17 antibody or antigen-binding fragment thereof. In preferred embodiments of the disclosed methods, treatments, regimens, uses and kits, the IL-17 antibody or antigen-binding fragment thereof is a human antibody of the IgG₁ isotype, with a k light chain. In preferred embodiments of the disclosed methods, the antibody or antigen-binding fragment thereof is secukinumab.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference as applicable, unless otherwise indicated. The following Examples are presented in order to more fully illustrate the preferred embodiments of the disclosure. These examples should in no way be construed as limiting the scope of the disclosed subject matter, which is defined by the appended claims.

### EXAMPLES

### Example 1—A Randomized, Parallel-Group, Double-Blind, Placebo-Controlled, Multicenter Phase III Trial to Investigate the Efficacy and Safety of Secukinumab 300 mg Administered Subcutaneously Versus Placebo, in Combination with a Glucocorticoid Taper Regimen, in Patients with Giant Cell Arteritis (GCA)

| | |
|---|---|
| Protocol number | CAIN457R12301 |
| Full title | A randomized, parallel-group, double-blind, placebo-controlled, |
| | multicenter phase 2 trial to investigate the safety and efficacy of |
| | secukinumab (AIN457) in patients with giant cell arteritis |
| | (TitAIN) |
| Brief title | TitAIN |
| Sponsor and | Novartis Pharma GmbH/ |
| Clinical phase | Phase II |
| Investigation type | Biological |
| Study type | Interventional |
| Purpose and rationale | This randomized, placebo-controlled, multicenter, Phase II trial |
| | in patients with newly diagnosed or relapsing giant cell arteritis |
| | (GCA) is proposed in order to investigate the potential effects of |
| | secukinumab in GCA. To date, no information on the safety and |
| | efficacy of secukinumab in GCA has been reported in a clinical |
| | trial setting. |
| Primary objective | The primary objective is to evaluate the efficacy of secukinumab |
| | compared to placebo, in combination with a 26-week |
| | prednisolone taper regimen, based on the proportion of patients |
| | with GCA who have sustained remission until Week 28 |
| | Definition of remission: absence of flare. |
| | Definition of sustained remission: patients without flare until |
| | Week 28 and in adherence to the protocol prednisolone taper |
| | regimen. |
| | Definition of flare: determined by the investigator and defined as |
| | the recurrence after remission of signs or symptoms of GCA |
| | and/or erythrocyte sedimentation rate (ESR) ≥ 30 mm/hr and/or |
| | CRP ≥ 10 mg/L attributable to GCA. |
| Secondary objectives | The secondary objectives are as follows: |
| | To evaluate the efficacy of secukinumab in combination with a 26-week |
| | prednisolone taper regimen versus placebo in patients with GCA, |
| | measured by the following: |
| | Remission rate at Week 12 |
| | Time to first flare after remission |
| | Cumulative corticosteroid dose up to Week 28 and up to Week 52 |
| | Patients in sustained remission up to Week 52 |
| | Definition of remission: absence of flare. |
| | Definition of sustained remission: patients without flare until |
| | Week 52 and in adherence to the protocol prednisolone taper |
| | regimen + prednisolone-free phase from Week 27 onwards. |
| | Definition of flare: determined by the investigator and defined |
| | as the recurrence after remission of signs or symptoms of GCA |
| | and/or erythrocyte sedimentation rate (ESR) ≥ 30 mm/hr and/or |
| | CRP ≥ 10 mg/L attributable to GCA. |
| | Proportion of patients on prednisolone dose ≤ 5mg/day at Week |
| | 19/Week 28/Week 52 |
| | Changes from baseline in disease activity and quality of life |
| | measures at Week 4, 8, 12, 16, 20, 24, 28, 36, 44 and 52 for each |
| | of the following: |
| | Physician's global assessment (PhGA) visual analog scale |
| | (VAS) |
| | Patient reported outcomes (PROs): |
| | Patient global assessment (PGA) VAS |
| | Functional Assessment of Chronic Illness Therapy |
| | Fatigue (FACIT-Fatigue) |
| | Short form 36 (SF36) |
| | EuroQoL 5D (EQ-5D) |
| | Laboratory parameters (Baseline vs. Week 4, 8, 12, 16, 20, 24, 28, 32, |
| | 36, 40, 44, 48 and 52): |
| | CRP |
| | ESR |
| | To evaluate the safety/tolerability and immunogenicity of secukinumab |
| | in patients with newly diagnosed or relapsing GCA. |
| Study design | This randomized, parallel-group, double-blind, placebo- |
| | controlled, multicenter, Phase II study is designed to evaluate |
| | the efficacy and safety of secukinumab compared to placebo in |
| | combination with a 26-week prednisolone taper regimen in |
| | terms of sustained remission in patients with newly diagnosed |
| | or relapsing GCA who are naive to biological therapy. |
| | The study will consist of a maximum 6-week screening period, a 52-week |
| | treatment period and a 8-week safety follow-up period. |
| | At Baseline, patients will be assigned to one of the following 2 arms in a 1:1 |
| | ratio: |
| | Group 1: secukinumab 300 mg s.c. + 26-week prednisolone taper |
| | regimen |
| | Group 2: placebo s.c. + 26-week prednisolone taper regimen |
| | Patients who do not achieve remission by Week 12, experience a flare after |
| | remission or cannot adhere to the prednisolone taper regimen will enter |
| | “escape”. Upon entering “escape”, patients will receive prednisolone at a |
| | dose determined by the investigator's clinical judgment and will continue to |
| | receive secukinumab or placebo in a blinded manner. Patients in “escape” |
| | should continue to attend all subsequent scheduled visit assessments. |
| Population | The study population will consist of 50 patients diagnosed with GCA (in |
| | accordance with inclusion criterion no. 4) and fulfilling all other entry criteria. |
| | Both patients with new onset GCA (diagnosed within 6 weeks of Baseline) |
| | and those with relapsing disease (diagnosed > 6 weeks of Baseline) will be |
| | included. Enrollment of patients with relapsing GCA will be preferentially |
| | limited to 50% but may be increased depending on the rate of enrollment of |
| | new-onset versus relapsing patients. |
| Inclusion criteria | Patients eligible for inclusion in this study must meet all of the |
| | following criteria: |
| | 1. Signed informed consent must be obtained prior to participation in |
| | the study. |
| | 2. Patient must be able to understand and communicate with the |
| | investigator and comply with the requirements of the study. |
| | 3. Male or non-pregnant, non-lactating female patients at least |
| | 50 years of age. |
| | 4. Diagnosis of GCA classified according to the following criteria: |
| | Age at onset of disease ≥ 50 years. |
| | History of ESR ≥ 30 mm/hr or CRP ≥ 10 mg/L. |
| | Unequivocal cranial symptoms of GCA (new-onset localized |
| | headache, scalp or temporal artery tenderness, ischemia- |
| | related vision loss, or otherwise unexplained mouth or jaw |
| | pain upon mastication) AND/OR symptoms of polymyalgia |
| | rheumatica (PMR) defined as shoulder and/or hip girdle pain |
| | associated with inflammatory morning stiffness. |
| | Temporal artery biopsy revealing features of GCA AND/OR |
| | evidence of large-vessel vasculitis by angiography or cross- |
| | sectional imaging study such as magnetic resonance |
| | angiography (MRA), computed tomography angiography (CTA), |
| | positron emission tomography-computed tomography |
| | (PET-CT), or ultrasound. |
| | 5. Patients with new onset GCA or relapsing GCA: |
| | Definition new onset: diagnosis of GCA within 6 weeks of |
| | Baseline Visit. |
| | Definition relapsing GCA: diagnosis of GCA (in accordance |
| | with inclusion criterion no. 4) > 6 weeks before Baseline Visit |
| | and in the meantime achieved remission (absence of signs |
| | and symptoms attributable to GCA and normalization of ESR |
| | (<30 mm/hr) and CRP (<10.0 mg/L) included) including |
| | previous treatment with ≥ 25 mg/day prednisolone equivalent |
| | for ≥ 2 weeks. |
| | 6. Active disease as defined by the presence of signs and symptoms |
| | of GCA (cranial or PMR) and elevated ESR ≥ 30 mm/hr, or |
| | CRP ≥ 10 mg/L, attributed to active GCA within 6 weeks of |
| | Baseline. |
| | 7. Prednisolone dose of 25-60 mg/day at Baseline. |
| | 8. Patients taking methotrexate (MTX) ≤ 25 mg/week are allowed to |
| | continue their medication provided they have taken it for at least 3 |
| | months and are on a stable dose for at least 4 weeks prior to |
| | randomization and if they are on a stable folic acid treatment before |
| | randomization. |
| Exclusion criteria | Patients who fulfill any of the following criteria are not eligible for |
| | inclusion in this study: |
| | 1. Pregnant or nursing (lactating) women where pregnancy is defined |
| | as the state of a female after conception and until the termination of |
| | gestation, confirmed by a positive human chorionic gonadotropin |
| | (hCG) laboratory test. |
| | 2. Women of childbearing potential, defined as all women |
| | physiologically capable of becoming pregnant, unless they are |
| | using effective methods of contraception during dosing of study |
| | treatment and for a minimum of 20 weeks after the last dose of |
| | secukinumab. Effective contraception methods include: |
| | Total abstinence (when this is in line with the preferred and |
| | usual lifestyle of the patient. Periodic abstinence (e.g. |
| | calendar, ovulation, symptothermal, post-ovulation methods) |
| | and withdrawal are not acceptable methods of contraception. |
| | Female sterilization (have had surgical bilateral |
| | oophorectomy with or without hysterectomy), total |
| | hysterectomy or tubal ligation at least 6 weeks before taking |
| | investigational drug. In case of oophorectomy alone, only |
| | when the reproductive status of the woman has been |
| | confirmed by follow-up hormone level assessment. |
| | Male sterilization (at least 6 months prior to screening). For |
| | female patients on the study, the vasectomized male partner |
| | should be the sole partner for that patient. |
| | Barrier methods of contraception: condom or occlusive cap |
| | (diaphragm or cervical/vault caps). |
| | Use of oral, (estrogen and progesterone), injected or |
| | implanted hormonal methods of contraception or other |
| | forms of hormonal contraception that have comparable |
| | efficacy. |
| | Women are considered post-menopausal and not of child bearing |
| | potential if they have had at least 12 months of natural |
| | (spontaneous) amenorrhea with an appropriate clinical profile (e.g. |
| | age appropriate, history of vasomotor symptoms) or have had |
| | surgical bilateral oophorectomy (with or without hysterectomy), |
| | total hysterectomy or tubal ligation at least 6 weeks ago. In the |
| | case of oophorectomy alone, only when the reproductive status |
| | of the woman has been confirmed by follow up hormone level |
| | assessment is she considered not of child bearing potential. |
| | 3. Previous exposure to secukinumab or other biologic drug directly |
| | targeting IL-17 or IL-17 receptor. |
| | 4. Patients treated with any cell-depleting therapies including but not |
| | limited to anti-CD20 or investigational agents (e.g. anti-CD3, anti- |
| | CD4, anti-CD5 or anti-CD19). |
| | 5. Patients who have previously been treated with any biologic agent |
| | including but not limited to tocilizumab, sirukumab, abatacept, or |
| | tumor necrosis factor alpha (TNFα) inhibitors (infliximab, |
| | adalimumab, etanercept, certolizumab, golimumab). |
| | 6. Patients who have previously been treated with tofacitinib or |
| | baricitinib. |
| | 7. Patients treated with i.v. immunoglobulins or plasmapheresis within |
| | 8 weeks prior to Baseline. |
| | 8. Patients treated with cyclophosphamide, tacrolimus or everolimus |
| | within 6 months prior to Baseline. |
| | 9. Patients treated with hydroxychloroquine, cyclosporine A, |
| | azathioprine, sulfasalazine or mycophenolate mofetil within 4 |
| | weeks of Baseline. |
| | 10. Patients treated with leflunomide within 8 weeks of Baseline unless |
| | a cholestyramine washout has been performed in which case the |
| | patient must be treated within 4 weeks of Baseline. |
| | 11. Patients treated with an alkylating agent except for |
| | cyclophosphamide as mentioned above. |
| | 12. Patients requiring systemic chronic glucocorticoid therapy for any |
| | other reason than GCA. |
| | 13. Chronic systemic glucocorticoid therapy over the last 4 years or |
| | longer; or inability, in the opinion of the investigator, to withdraw |
| | glucocorticoid therapy through protocol-defined taper regimen due |
| | to suspected or established adrenal insufficiency. |
| | 14. Patients requiring chronic (i.e. not occasional “prn”) high potency |
| | opioid analgesics for pain management. |
| | 15. Patients treated with any investigational agent within 4 weeks or |
| | within 5 half-lives of the drug (whichever is longer) prior to Baseline. |
| | 16. Contraindication or hypersensitivity to secukinumab. |
| | 17. Active ongoing inflammatory diseases other than GCA that might |
| | confound the evaluation of the benefit of secukinumab therapy. |
| | 18. Active ongoing inflammatory diseases or underlying metabolic, |
| | hematologic, renal, hepatic, pulmonary, neurologic, endocrine, |
| | cardiac, infectious or gastrointestinal conditions, which in the |
| | opinion of the investigator immunocomprises the patient and/or |
| | places the patient at unacceptable risk for participation in an |
| | immunomodulatory therapy. |
| | 19. Significant medical problems or diseases, including but not limited |
| | to the following: uncontrolled hypertension (≥160/95 mmHg), |
| | congestive heart failure (New York Heart Association (NYHA) |
| | status of class III or IV) and uncontrolled diabetes. |
| | 20. History of clinically significant liver disease or liver injury as |
| | indicated by abnormal liver function tests (LFTs) such as aspartate |
| | aminotransferase (AST), alanine aminotransferase (ALT), alkaline |
| | phosphatase (ALP) or serum bilirubin. The investigator should be |
| | guided by the following criteria: |
| | a. Any single parameter may not exceed 2 × upper limit of |
| | normal (ULN). A single parameter elevated up to and |
| | including 2 × ULN should be re-checked once more as soon |
| | as possible and in all cases, at least prior to randomization, |
| | to rule out any possible laboratory error. |
| | b. If the total bilirubin concentration is increased above 2 × ULN, |
| | total bilirubin should be differentiated into the direct and |
| | indirect reacting bilirubin. |
| | 21. History of renal trauma, glomerulonephritis, or patients with one |
| | kidney only, or a serum creatinine level exceeding 1.8 mg/dL |
| | (159.12 μmol/L). |
| | 22. Screening total white blood cell (WBC) count < 3000/μL, or platelets < |
| | 100 000/μL or neutrophils < 1500/μL or hemoglobin < 8.3 g/dL |
| | (83 g/L). |
| | 23. Active systemic infections during the last 2 weeks (exception: |
| | common cold) prior to randomization. |
| | 24. Major ischemic event, unrelated to GCA, within 12 weeks of |
| | screening. |
| | 25. Any major infection requiring oral antibiotic treatment within |
| | 2 weeks prior to Baseline. |
| | 26. Major surgery within 8 weeks prior to screening or planned major |
| | surgery within 12 months after randomization. |
| | 27. History of ongoing, chronic or recurrent infectious disease or |
| | evidence of tuberculosis infection as defined by either a positive |
| | purified protein derivative (PPD) skin test (the size of induration will |
| | be measured after 48-72 hours, and a positive result is defined as |
| | an induration of ≥ 5 mm or according to local practice/guidelines), or |
| | a positive QuantiFERON TB-Plus test. Patients with a positive test |
| | may participate in the study if further work up (according to local |
| | practice/guidelines) establishes conclusively that the patient has no |
| | evidence of active tuberculosis. If presence of latent tuberculosis is |
| | established then treatment according to local country guidelines |
| | must be initiated prior to randomization. |
| | 28. Known infection with human immunodeficiency virus (HIV), |
| | hepatitis B or hepatitis C at screening or randomization. |
| | 29. History of lymphoproliferative disease or any known malignancy or |
| | history of malignancy of any organ system within the past 5 years |
| | (except for basal cell carcinoma or actinic keratoses that have been |
| | treated with no evidence of recurrence in the past 3 months, |
| | carcinoma in situ of the cervix or non-invasive malignant colon |
| | polyps that have been removed). |
| | 30. Life vaccinations within 6 weeks prior to Baseline or planned |
| | vaccination during study participation until 12 weeks after last study |
| | treatment administration. |
| | 31. Current severe progressive or uncontrolled disease which in the |
| | judgment of the clinical investigator renders the patient unsuitable |
| | for the trial. |
| | 32. Inability or unwillingness to undergo repeated venipuncture |
| | (e.g. because of poor tolerability or lack of access to veins). |
| | 33.Any medical or psychiatric condition which in the investigator's |
| | opinion, would preclude the participant from adhering to the |
| | protocol or completing the study per protocol. |
| | 34. Donation or loss of 400 mL or more of blood within 8 weeks before |
| | randomization. |
| | 35. History or evidence of ongoing alcohol or drug abuse, within the |
| | last 6 months before randomization. |
| | No additional exclusions may be applied by the investigator, in order to |
| | ensure that the study population will be representative of all eligible patients. |
| Study treatment | Investigational drug: secukinumab 300 mg supplied in prefilled |
| | syringes (PFS) each containing 150 mg of secukinumab. |
| | Control: placebo supplied in PFS to match secukinumab dose |
| | Co-administered treatment: prednisolone tablets for tapered oral |
| | administration (taper regimen from a dose of 25 mg to 60 mg at Baseline to |
| | 0 mg at Week 27. Prednisolone will be supplied as 1 mg, 5 mg, 10 mg, 20 |
| | mg tablets. |
| Efficacy assessments | GCA assessment (signs and symptoms) |
| | PhGA |
| | ESR |
| | CRP |
| | Patient reported outcomes: |
| | PGA |
| | EQ-5D |
| | SF-36 |
| | FACIT-Fatigue |
| Immunogenicity | Immunogenicity assessment of anti-AIN457 antibodies. |
| assessment | |
| Safety assessments | Physical examination |
| | Vital signs |
| | Laboratory parameters |
| | Adverse events (AEs)/serious adverse events (SAEs) |
| Data analysis | The primary endpoint is the proportion of GCA patients who adhere to the |
| | prednisolone taper regimen and are in sustained remission until Week 28. |
| | The response rate of the comparable placebo-arm of the GIACTA study will |
| | be used as the prior distribution for the placebo response rate for the primary |
| | endpoint in this study. The prior distribution for the response rate on |
| | secukinumab will be a uniform Beta distribution. |
| | Posterior distributions for the estimate of the odds ratio, risk-ratio and risk |
| | difference will be derived by sampling from the posterior distributions of the |
| | response rates of secukinumab and placebo. |

### Objectives and Related Endpoints

TABLE 1
Objectives and related endpoints

| Objectives | Endpoints |
|---|---|
| Primary objective | Endpoint for primary objective |
| To evaluate the efficacy of secukinumab | Proportion of GCA patients in sustained |
| compared to placebo, in combination with a | remission at Week 28. |
| 26-week prednisolone taper regimen, based on | |
| the proportion of patients with GCA who have | |
| sustained remission. | |
| Definition of remission: Absence of flare. | |
| Definition of sustained remission: patients | |
| without flare until Week 28 and in | |
| adherence to the protocol prednisolone | |
| taper regimen. | |
| Definition of flare: Determined by the | |
| investigator and defined as the recurrence | |
| after remission of signs or symptoms of | |
| GCA and/or ESR ≥ 30 mm/hr and/or | |
| CRP ≥ 10 mg/L attributable to GCA. | |
| Secondary objectives: | Endpoint for secondary objectives: |
| To evaluate the efficacy of secukinumab in | |
| combination with a 26-week prednisolone taper | |
| regimen versus placebo in patients with GCA, | |
| measured by the following: | |
| Remission rate at Week 12 | Remission rate at Week 12 |
| Time to first flare after remission | Time to first GCA flare after clinical |
| | remission (up to Week 52) |
| | Total cumulative prednisolone dose over 28 |
| | weeks and 52 weeks |
| Cumulative corticosteroid dose up to Week 28 | Proportion of GCA patients in sustained |
| and up to Week 52 | remission at Week 52 |
| Proportion of patients with GCA who have | |
| sustained remission | |
| Definition of remission: absence of flare. | |
| Definition of sustained remission: patients | |
| without flare until Week 52 and in | |
| adherence to the protocol prednisolone | |
| taper regimen + prednisolone-free phase | |
| from Week 27 onwards. | |
| Definition of flare: determined by the | |
| investigator and defined as the recurrence | |
| after remission of signs or symptoms of | |
| GCA and/or erythrocyte sedimentation rate | |
| (ESR) ≥ 30 mm/hr and/or CRP ≥ 10 mg/L | |
| attributable to GCA. | |
| Proportion of patients on prednisolone | |
| dose ≤ 5 mg/day at Week 19/Week 28/Week 52 | Proportion of patients on prednisolone |
| | dose ≤ 5 mg/day at Week 19, Week 28 and Week |
| | 52 |
| Changes from baseline in disease activity | Changes from Baseline in disease activity |
| and quality of life measures at Week 4, 8, | and quality of life measures at Week 4, 8, |
| 12, 16, 20, 24, 28, 36, 44 and 52 for each | 12, 16, 20, 24, 28, 36, 44 and 52 for each of |
| of the following: | the following: |
| PhGA, VAS | PhGA, VAS |
| PRoS: | PRoS: |
| PGA, VAS | PGA, VAS |
| FACIT-Fatigue | FACIT-Fatigue |
| SF36 | SF-36 |
| EQ-5D | EQ-5D |
| Laboratory parameters | Change from Baseline in CRP and ESR at |
| (Baseline vs. Week 28 and 52): | Week 4, 8, 12, 16, 20, 24, 28, 32, 36, 40, 44, |
| CRP | 48 and 52 |
| ESR | |
| To evaluate the safety/ tolerability and | Safety and tolerability assessments over |
| immunogenicity of secukinumab in patients with | time: incidence and severity of AEs and |
| newly diagnosed or relapsing GCA. | SAEs; routine safety laboratory parameters |

### Investigational Plan

### Study Design

This randomized, parallel-group, double-blind, placebo-controlled, multicenter, Phase II study is designed to evaluate the efficacy of secukinumab compared to placebo in combination with a 26-week prednisolone taper regimen in terms of sustained remission in patients with newly diagnosed or relapsing GCA who are naïve to biological therapy.

The study will consist of 6-week (maximum duration) screening period, a 52-week treatment period and a 8-week safety follow-up period (Error! Reference source not found.). Patients who do not achieve remission by Week 12, experience a flare after remission or cannot adhere (as described below) to the prednisolone taper regimen will enter “escape”. Upon entering “escape”, patients will receive prednisolone at a dose determined by the physician's clinical judgment and continue to receive secukinumab or placebo in a blinded manner. Patients in “escape” should continue to attend all subsequent scheduled visit assessments.

Patients who have received an additional corticosteroid as stated in the taper regimen will be considered as not having adhered to the protocol-defined prednisolone taper regimen. Patients not adhering to the prednisolone taper will be classified as non-responders in the primary analysis, regardless of their status of sustained remission. Patients receiving less than the full amount of prednisolone as required by the taper (due to missing tablets) will not be classified as non-responders, unless other non-response criteria are fulfilled.

Screening period: During the screening period, patients may receive glucocorticoids for the treatment of GCA at the discretion of the investigator. By the end of the screening period patients should be able to switch to the Sponsor-provided prednisolone in order to follow the protocol-defined prednisolone tapering regimens.

Randomization: Patients will be assigned to one of the following 2 arms in a 1:1 ratio:
Group 1: secukinumab 300 mg s.c.+26-week prednisolone taper regimen
Group 2: placebo s.c.+26-week prednisolone taper regimen

Patients will receive secukinumab/placebo at Baseline, Week 1, 2, 3, 4, then every 4 weeks thereafter through to Week 48 (last dose) at the study center. Assessments will be performed in accordance to the study schedule. Patients in both groups will follow the protocol-defined prednisolone taper regimen.

At every visit, each patient's disease activity and condition will be assessed in order to determine whether the patient can adhere to the defined prednisolone taper regimen (open-label fashion). If they cannot adhere to the taper regimen or are experiencing a flare (definition above), they must enter the “escape” arm and receive prednisolone at a dose determined by the physician based on clinical need and will continue to receive secukinumab 300 mg or placebo s.c. (still blinded).

Safety evaluation will be included in all visits including two safety follow-up visits performed 8 and 12 weeks after the last study drug administration at Week 48 (this will be at Week 56 and Week 60 for patients completing the study according to the protocol).

For the primary analysis, a treatment duration of 24 weeks was selected for this study. This is shorter than the 52-week treatment duration in the GiACTA trial (Stone et al 2017); however, most flares occurred within the first 6 months in the GiACTA trial. Therefore, the time point for primary analysis is at Week 28, to evaluate the effect of secukinumab in patients are on study treatment or placebo for 24 weeks. Assessments for the primary analysis will be conducted at Week 28, 2 weeks after last prednisolone dose in Week 26.

Secondary endpoints address both assessments at Week 28, after 24 weeks of treatment including a 26-week-prednisolone taper and also assessments at Week 52, after a total of 48 weeks of treatment, without concomitant prednisolone from Week 27 onwards.

From Week 27 up to Week 52, it will be assessed whether secukinumab is effective to provide sustained remission, decreased vessel involvement (assessed by imaging) and improved quality of life without concomitant prednisolone. Patients will receive double-blind treatment consisting of two injections per time point up to Week 48, final efficacy assessments will be performed at Week 52.

Regular assessments ensure that the patients who may experience a flare in any of the treatment groups can enter the escape arm in order to receive prednisolone based on the investigator's clinical judgement.

The 8-week follow-up period after the end of study treatment is included to generate follow-up data for potential relapse and to have treatment safety follow-up data in the GCA indication. The 26-week prednisolone taper regimen was selected as this proved to be sufficiently effective in combination with tocilizumab for achieving sustained glucocorticoid-free remission in patients with GCA in the GIACTA-trial (Stone et al 2017). Pivotal Phase III trials of secukinumab in other chronic inflammatory diseases (e.g. psoriasis, PsA) have demonstrated peak efficacy (i.e. remission of disease) at Week 12, which has been sustained until Week 24. In this Phase II trial, those patients who do not achieve remission at Week 12 will be entered into an “escape” treatment arm where they will continue to receive secukinumab or placebo in a blinded fashion but their prednisolone treatment may be adjusted by the investigator based on their clinical needs.

The study population will consist of 50 patients diagnosed with GCA (in accordance with inclusion criterion no. 4) and fulfilling all other entry criteria. Both patients with new onset GCA (diagnosed within 6 weeks of Baseline) and those who have relapsing disease (diagnosed >6 weeks before Baseline) will be included. Enrollment of patients with relapsing GCA will be preferentially limited to 50% but may be increased depending on the rate of enrollment of new-onset versus relapsing patients.

### Study Treamement

### Investigational and Control Drugs

An overview of study treatment is presented in Table. Patients will be given all secukinumab and placebo injections by the site staff on site. The PFSs are packed in a double-blind fashion and do not need to be prepared. The study treatments will be labeled as follows:
Double-blind secukinumab and placebo PFSs will be labeled AIN457 150 mg/1 ml/Placebo for dosing up to and including Week 48.

Patients will also receive open-label prednisolone tablets for tapered dosing from Baseline to Week 26. Patients will be given prednisolone tablets by the site staff on site at study site visits, and will be supplied with sufficient tablets for home administration between study visits.

TABLE 2
Overview of study treatment

| Study drug name | Formulation | Unit dose | Packaging | Provided by |
|---|---|---|---|---|
| Secukinumab 300 mg | 2 × 1 mL PFS | 1 mL | Double-blind supply | Novartis |
| (2 injections of 150 mg) | | (150 mg) | | |
| Placebo, 2 injections | 2 × 1 mL PFS | | Double-blind supply | Novartis |
| Prednisolone, (taper | Tablets for oral | 1 mg, 5 mg, | Open-label supply | Novartis |
| regimen from a dose of | administration | 10 mg, | | |
| 25 mg to 60 mg at | | 20 mg | | |
| Baseline to 1 mg at | | | | |
| Week 26 [last dose]) | | | | |

### Additional Treatment

Prednisolone will be administered alongside the investigational drug (secukinumab) and placebo in this trial as described herein. No further additional treatment is included in this trial.

### Treatment Arms

Patients will be assigned to one of the following 2 treatment arms in a 1:1 ratio, with approximately 25 patients per arm.
Group 1: Secukinumab 300 mg s.c. (2×150 mg)+26-week prednisolone taper regimen
Group 2: Placebo s.c. (2 injections)+26-week prednisolone taper regimen

Patients will receive secukinumab or placebo at Week 0 (Baseline), Week 1, 2, 3, 4, then every 4 weeks thereafter through to Week 48 (last dose) at the study center.

Patients will receive a daily dose of prednisolone, which will be decreased (i.e. tapered down) from Baseline to Week 26.

Patients who do not achieve remission by Week 12, experience a flare after remission or cannot adhere to the prednisolone taper regimen will enter “escape”. Upon entering “escape”, patients will receive prednisolone at a dose determined by the physician's clinical judgment and continue to receive secukinumab 300 mg s.c. in a double-blind manner. Patients in “escape” should continue to attend all subsequent scheduled visit assessments.

Patients who have received an additional corticosteroid as stated in the taper regimen will be considered as not having adhered to the protocol-defined prednisolone taper regimen. Patients not adhering to the prednisolone taper will be classified as non-responders in the primary analysis, regardless of their status of sustained remission. Patients receiving less than the full amount of prednisolone as required by the taper (due to missing tablets) will not be classified as non-responders.

TABLE 3
Study treatment schedule

| Investigational/Control Drug | | Frequency and/or |
|---|---|---|
| (Name and Strength) | Dose | Regimen |
| Secukinumab 300 mg | | Week 0 to 4: weekly |
| (2 injections of 150 mg) | 300 mg (2 × 1 mL PFS each | From Week 4 to |
| | containing 150 mg secukinumab) | Week 48: every |
| | | 4 weeks |
| Placebo, 2 injections | Not applicable. | As above. |
| Prednisolone, | | |
| (taper regimen from a dose of 25 mg to | Tapered from Baseline to Week 26 | Daily |
| 60 mg at Baseline to 1 mg at Week 26 | (see Table 0-1 and Table) | |
| [last dose]) | | |

Two different prednisolone taper regimens (Table 0-1 for patients on 40 to 60 mg/day prednisolone at Baseline; and Table for patients on 25 to 40 mg/day prednisolone at Baseline) will be used for both treatment arms depending on patients' prednisolone levels at Baseline. From Week 8, all patients will receive the same prednisolone level (15 mg/day) and will continue to be tapered down. Prednisolone will be supplied by the Sponsor in an open-label fashion. Prednisolone intakes at weeks, which are not part of the assessment schedule need to be reported (date/, home administration form).

TABLE 0-1
60-40 mg/day at Baseline

| | | Prednisolone | | | | |
|---|---|---|---|---|---|---|
| | Dose | 20 mg, | 10 mg, | 5 mg, | 1 mg, | |
| Week | mg/day | n(tablets) | n(tablets) | n(tablets) | n(tablets) | n/visit |
| 0 | 60-40 | Supplied by sponsor, patients start tapering | | | | |
| | | on individual level within the ranges, from | | | | |
| 1 | 55-35 | Week 7 in accordance with the taper regimen | | | | |
| 2 | 50-30 | | | | | |
| 3 | 45-28 | | | | | |
| 4 | 35-25 | | | | | |
| 5 | 30-22 | | | | | |
| 6 | 25-21 | | | | | |
| 7 | 20 | | 2 | | | 2 |
| 8 | 15 | | 1 | 1 | | 2 |
| 9 | 13 | | 1 | | 3 | 4 |
| 10 | 12 | | 1 | | 2 | 3 |
| 11 | 10 | | | 2 | | 2 |
| 12 | 9 | | | 1 | 4 | 5 |
| 13 | 8 | | | 1 | 3 | 4 |
| 14 | 7 | | | 1 | 2 | 3 |
| 15 | 6 | | | 1 | 1 | 2 |
| 16 | 6 | | | 1 | 1 | 2 |
| 17 | 5 | | | 1 | | 1 |
| 18 | 5 | | | 1 | | 1 |
| 19 | 4 | | | | 4 | 4 |
| 20 | 4 | | | | 4 | 4 |
| 21 | 3 | | | | 3 | 3 |
| 22 | 3 | | | | 3 | 3 |
| 23 | 2 | | | | 2 | 2 |
| 24 | 2 | | | | 2 | 2 |
| 25 | 1 | | | | 1 | 1 |
| 26 | 1 | | | | 1 | 1 |
| 27 | 0 | | | | | |
| 28 | 0 | | | | | |

Grey: no site visit

TABLE 4
40-25 mg/day at Baseline

| | | Prednisolone | | | | |
|---|---|---|---|---|---|---|
| | Dose | 20 mg, | 10 mg, | 5 mg, | 1 mg, | |
| Week | mg/day | n(tablets) | n(tablets) | n(tablets) | n(tablets) | n/visit |
| 0 | 40-25 | Supplied by sponsor, patients start tapering | | | | |
| 1 | 35-22 | on individual level within the ranges, from | | | | |
| 2 | 30-21 | week 7 in accordance with the taper regimen | | | | |
| 3 | 27-20 | | | | | |
| 4 | 25-19 | | | | | |
| 5 | 22-18 | | | | | |
| 6 | 20-17 | | | | | |
| 7 | 16 | | 1 | 1 | 1 | 3 |
| 8 | 15 | | 1 | 1 | | 2 |
| 9 | 13 | | 1 | | 3 | 4 |
| 10 | 12 | | 1 | | 2 | 3 |
| 11 | 10 | | | 2 | | 2 |
| 12 | 9 | | | 1 | 4 | 5 |
| 13 | 8 | | | 1 | 3 | 4 |
| 14 | 7 | | | 1 | 2 | 3 |
| 15 | 6 | | | 1 | 1 | 2 |
| 16 | 6 | | | 1 | 1 | 2 |
| 17 | 5 | | | 1 | | 1 |
| 18 | 5 | | | 1 | | 1 |
| 19 | 4 | | | | 4 | 4 |
| 20 | 4 | | | | 4 | 4 |
| 21 | 3 | | | | 3 | 3 |
| 22 | 3 | | | | 3 | 3 |
| 23 | 2 | | | | 2 | 2 |
| 24 | 2 | | | | 2 | 2 |
| 25 | 1 | | | | 1 | 1 |
| 26 | 1 | | | | 1 | 1 |
| 27 | 0 | | | | | |
| 28 | 0 | | | | | |

grey: no site visit

### Prednisolone Prior and Up to Baseline

Patients must be on prednisolone dose of 25-60 mg/day at Baseline in order to be included in the study.

Prednisolone from Baseline

Patients will receive a daily dose of prednisolone, which will be decreased (i.e. tapered down) from Baseline to Week 26 as described.

### Methotrexate

Patients taking MTX (≤25 mg/week) at study entry are allowed to continue their medication provided they have taken it for at least 3 months and are on a stable dose for at least 4 weeks prior to randomization and throughout the study.

Patients on MTX must be taking folic acid supplementation before randomization and during the trial to minimize the likelihood of MTX associated toxicity.

Leflunomide Wash-Out with Cholestyramine

In case of leflunomide treatment, a drug wash-out of 8 weeks has to be performed; however, another wash-out procedure may be considered. Cholestyramine can be given orally at a dose of 8 g three times daily to wash out leflunomide. Cholestyramine has been shown to reduce plasma levels of the active leflunomide metabolite by approximately 40% in 24 hours and by 49% to 65% in 48 hours in 3 healthy volunteers. The administration of cholestyramine is recommended in patients who require a drug elimination procedure. If a patient receives 8 g three times daily for 11 days he/she can be safely randomized 4 weeks after the beginning of the 11-day cholestyramine treatment period.

### Vitamin D

Vitamin D (1000 I.E. per day) is strongly recommended during the study.

### Efficacy

### Primary Efficacy Assessment

The primary efficacy objective is to evaluate the efficacy of secukinumab compared to placebo, in combination with a 26-week prednisolone taper regimen, based on the proportion of patients with GCA who have sustained remission.

The primary efficacy endpoint is the proportion of GCA patients in sustained remission (as defined below) at Week 28.

Definition of remission: Absence of flare (see below). Definition of sustained remission: patients without flare (see below) until Week 28 and in adherence to the protocol prednisolone taper regimen.

Definition of flare: Determined by the investigator and defined as the recurrence after remission of signs or symptoms of GCA and/or ESR≥30 mm/hr and/or CRP≥10 mg/L attributable to GCA.

### Signs and Symptoms of GCA Disease

Evaluation of clinical signs and symptoms by the Efficacy Assessor at every study visit according to the schedule of assessment will include the following:
Fever (>38° C.).
Symptoms of polymyalgia rheumatica (PMR) (morning stiffness and/or pain, in the shoulder and/or hip girdles).
Localized headache, temporal artery or scalp tenderness.
Visual signs or symptoms such as acute or subacute vision loss due to arteritic anterior ischemic optic neuropathy (A-AION), transient blurry vision (generally monocular or at least affecting one eye at a time, but potentially affecting both eyes).
Jaw or mouth pain.
New or worsened extremity claudication.
Other features judged by the clinician-investigator to be consistent with a GCA or PMR flare.

### Secondary Efficacy Assessments

The following secondary efficacy endpoints will be assessed as herein described or described in the respective protocol sections:
Remission rate at Week 12
Time to first GCA flare after clinical remission (up to Week 52
Total cumulative prednisolone dose up to Weeks 28 and 52
Proportion of GCA patients in sustained remission at Week 52
   Definition of remission: absence of flare.
   Definition of sustained remission: patients without flare until Week 52 and in adherence to the protocol prednisolone taper regimen+prednisolone-free phase from Week 27 onwards.
   Definition of flare: determined by the investigator and defined as the recurrence after remission of signs or symptoms of GCA and/or erythrocyte sedimentation rate (ESR)≥30 mm/hr and/or CRP≥10 mg/L attributable to GCA.
Proportion of patients on prednisolone dose <5 mg/day at Week 19, Week 28 and Week 52
Changes from Baseline in disease activity and quality of life measures at Week 4, 8, 12, 16, 20, 24, 28, 36, 44, 52 for each of the following:
   PhGA, VAS.
   PROs:
      PGA, VAS
      FACIT-Fatigue
      SF-36
      EQ-5D
Change from Baseline at Week 4, 8, 12, 16, 20, 24, 28, 32, 36, 40, 44, 48 and 52 in CRP and ESR

All efficacy assessments should be performed prior to administration of study treatment.

The primary efficacy assessment in this study is aligned with the primary efficacy assessment in the GIACTA trial (Stone et al 2017), which demonstrated the efficacy of another humanized monoclonal antibody (i.e. tocilizumab) in combination with similar corticosteroid tapering (i.e. prednisone) for the treatment of patients with GCA. Other efficacy assessments, including the PhGA and patient reported outcome (PRO) assessments, including PGA are closely aligned with those included in other Phase II and Phase III clinical trials in the clinical development program for secukinumab for other chronic inflammatory diseases.

The patient should be given the PRO measures to be completed at the scheduled visit before any clinical assessments are conducted. The patient should be given sufficient space and time to complete the PRO measures.

The site personnel (referring to who could be responsible for administering and checking completions of PRO measures) should check PRO measures for completeness and ask the patient to complete any missing responses. The responses stored electronically on the database will be considered the source file.

Completed measures and any unsolicited comments written by the patient should be reviewed and assessed by the investigator for responses, which may indicate potential AEs or SAEs before any clinical study examinations. This assessment should be documented in study source records. If AEs or SAEs are confirmed, study investigators should not encourage the patient to change responses reported in the completed questionnaires. Study investigators must follow reporting instructions outlined in the study protocol.

### Patient's Global Assessment of Disease Activity

The patient's global assessment of disease activity will be performed using 100 mm VAS ranging from “has no effect at all” to “worst possible effect”, after the question “On a scale of 0-100 where would you rate the overall effect your Giant Cell Arteritis has on you at this time”.

The patient's global assessment of disease activity is filled in by the patient at all visits specified prior to study drug administration.

The FACIT-Fatigue© is a 13-item questionnaire (Cella 1993 and Yellen 1997) that assesses self-reported fatigue and its impact upon daily activities and function.

The purpose of collecting available FACIT-Fatigue© data according to clinical routine of the Treating Physician in the eCRF is to assess the impact of fatigue on patients with GCA. The questionnaire will be used at all visits specified prior to study drug administration.

The SF-36 is a widely used and extensively studied instrument to measure health-related quality of life among healthy subjects and patients with acute and chronic conditions. It consists of eight subscales that can be scored individually: Physical Functioning, Role-Physical, Bodily Pain, General Health, Vitality, Social Functioning, Role-Emotional, and Mental Health (Ware 1993). Two overall summary scores, the Physical Component Summary (PCS) and the Mental Component Summary (MCS) also can be computed (Ware 1994). The SF-36 has proven useful in monitoring general and specific populations, comparing the relative burden of different disease, differentiating the health benefits produced by different treatments, and in screening individual patients.

The purpose of the SF-36 in this study is to assess the health-related quality of life of patients. The standard version with a 4-week recall period, will be used at all visits specified prior to study drug administration.

The EQ-5D-5L is a widely used, self-administered questionnaire designed to assess health status in adults (Xu et al 2011, Mc Clure et al 2017). The purpose of the EQ-5D-5L in this study is to assess the general health status of the patients. The measure is divided into 2 distinct sections. The first section includes 1 item addressing each of 5 dimensions (mobility, self-care, usual activity, pain/discomfort, and anxiety/depression). Patients rate each of these items from “no problem”, “slight problems”, “moderate problems”, “severe problems”, “extreme problems/unable.” A composite health index is then defined by combining the levels for each dimension. The second section of the questionnaire measures self-rated (global) health status utilizing a vertically oriented VAS where 100 represents the “best possible health state” and 0 represents the “worst possible health state.” Respondents are asked to rate their current health by placing a mark along this continuum. The recall period is “today,” and the questionnaire requires approximately 5 to 10 minutes to complete.

The EQ-5D-5L contains 6 items designed to assess health status in terms of a single index value or health utility score. One of the strengths of the EQ-5D-5L approach is that it allows “weighting” by the patient of particular health states and the generation of patient utilities. Published weights are available that allow for the creation of a single summary health utility score. Overall scores range from 0 to 1, with lower scores representing a higher level of dysfunction. The EQ-5D-5L is filled in by the patient at the visits specified prior to study drug administration.

### Results

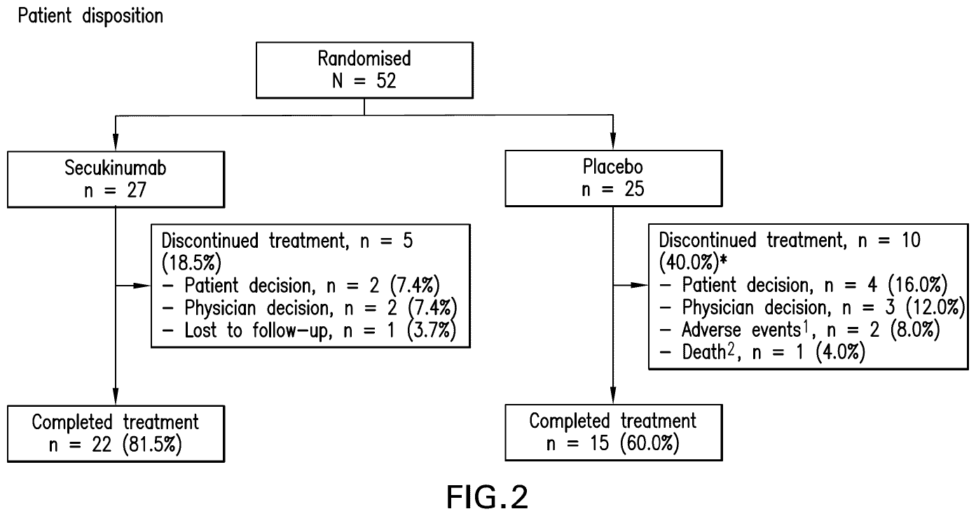

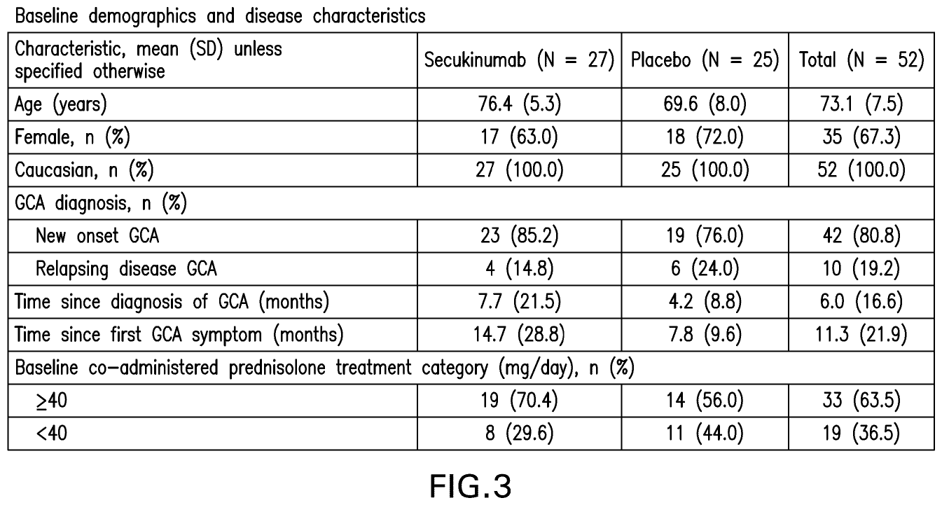

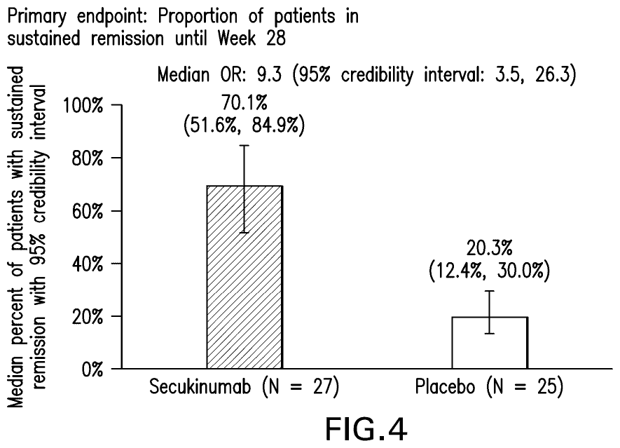

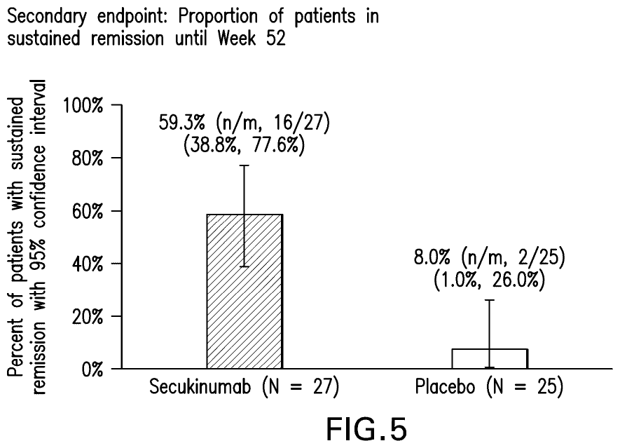

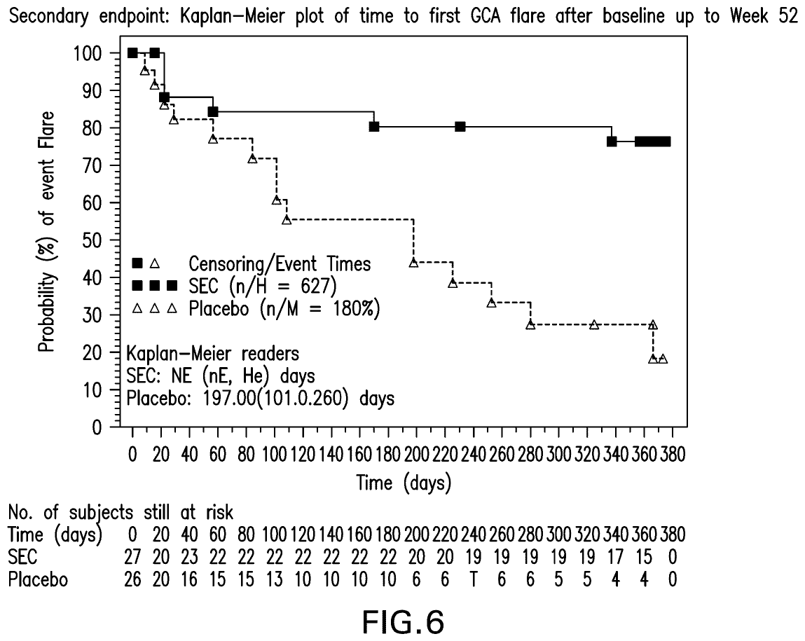

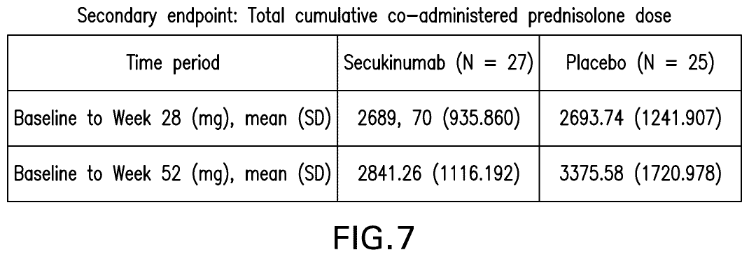

The results are shown and described in FIGS. 2 to 7. Out of 52 randomized patients (SEC, n=27; placebo, n=25), 71.2% (n=37) completed study treatment (SEC, 81.5%; placebo, 60.0%). Overall, 42 (80.8%) patients had new onset GCA and 10 (19.2%) patients had relapsing GCA at baseline. The proportion (posterior median with 95% credibility interval) of GCA patients in sustained remission until Week 28 was higher with SEC, 70.1% (51.6%-84.9%), than with placebo, 20.3% (12.4%-30.0%); odds ratio (posterior median with 95% credibility interval), 9.31 (3.54-26.29) Until Week 52, the proportion (95% confidence interval) of GCA patients in sustained remission were 59.3% (38.8%-77.6%) in SEC group and 8.0% (1.0%-26.0%) in placebo group. The median (95% confidence interval) time to first GCA flare after baseline was not reached for GCA patients treated with SEC and was 197.0 (101.0-280.0) days for placebo.

### Example 2—A Randomized, Parallel-Group, Double-Blind, Placebo-Controlled, Multicenter Phase 2 Trial to Investigate the Safety and Efficacy of Secukinumab (AIN457) in Patients with Giant Cell Arteritis (TitAIN)

| | |
|---|---|
| Primary | The primary objective is to demonstrate that the efficacy of secukinumab 300 mg s.c. |
| Objective(s) | in combination with a 26-week Glucocorticoids (GC) taper regimen is superior to |
| | placebo in combination with a 52-week GC taper regimen in participants with GCA |
| | based on sustained remission at Week 52. |
| Secondary | To assess the efficacy of secukinumab in combination with a 26-week GC taper |
| Objectives | regimen compared to placebo in combination with a 52-week GC taper regimen in |
| | participants with GCA based on time to clinical failure through Week 52 |
| | To assess the efficacy of secukinumab in combination with a 26-week GC taper |
| | regimen compared to placebo in combination with a 52-week GC taper regimen, |
| | in participants with GCA, based on cumulative GC dose through Week 52 |
| | To assess the effect on participant's QoL of secukinumab in combination with a |
| | 26-week GC taper regimen compared to placebo in combination with a 52-week |
| | GC taper regimen, in participants with GCA based on change of SF-36 score |
| | [Physical Component Summary (PCS)] at Week 52 |
| | To assess the effect of secukinumab in combination with a 26-week GC taper |
| | regimen compared to placebo in combination with a 52-week GC taper regimen, |
| | in participants with GCA based on change in Glucocorticoid Toxicity Index |
| | (GTI) at Week 52 |
| | To assess the effect on participant's QoL of secukinumab in combination with a |
| | 26-week GC taper regimen compared to placebo in combination with a 52-week |
| | GC taper regimen, in participants with GCA based on change in FACIT-Fatigue |
| | score at Week 52 |
| | To assess the effect on participant's QoL of secukinumab treatment in |
| | combination with a 26-week GC taper regimen compared to placebo in |
| | combination with a 52-week GC taper regimen, in participants with GCA based on |
| | change in EQ-5D score at Week 52 |
| | To evaluate the safety and tolerability of secukinumab |
| Study design | This randomized, parallel-group, double-blind, placebo-controlled, multicenter, Phase |
| | Ill study is designed to evaluate the efficacy of secukinumab in combination with a 26- |
| | week prednisone taper regimen compared to placebo in combination with a 52-week |
| | prednisone taper regimen based on sustained remission at Week 52 in participants with |
| | newly diagnosed or relapsing GCA. |
| | The study will consist of 6-week screening period, a 56-week treatment period |
| | (Treatment Period 1), a second treatment period of 52 weeks (Treatment Period 2) and |
| | an 8-week safety Follow-Up Period. |
| | Screening period: During the screening period, it is expected that participants will be |
| | treated for GCA with GCs as per the investigator's judgement. By the end of the |
| | screening period participants should be able to switch to the sponsor-provided |
| | prednisone at Baseline in order to follow the protocol-defined prednisone tapering |
| | regimens. Starting prednisone doses at Baseline are 20, 25, 30, 35, 40, 50 or 60 mg/day |
| | at the investigator's discretion. |
| | Treatment Period 1 (Baseline (BSL) to Week 56 pre-dose): At Baseline, participants |
| | will be randomized to one of the following 2 arms in a 2:1 ratio: |
| | Group 1: Secukinumab 300 mg (2 mL PFS) s.c. at BSL, Weeks 1, 2, 3, followed by |
| | administration every four weeks starting at Week 4. Secukinumab will be given in |
| | combination with a specified 26-week prednisone taper regimen |
| | Group 2: Placebo to secukinumab 300 mg (2 mL PFS) s.c. at BSL, Weeks 1, 2, 3, |
| | followed by administration every four weeks starting at Week 4. Placebo will be given |
| | in combination with a specified 52-week prednisone taper regimen. |
| | Randomization in the two groups will be stratified by the participant's baseline |
| | GCA status (new-onset or relapsing GCA) and prednisone dose (≤30 mg/day or >30 |
| | mg/day prednisone). |
| | After Baseline, participants who do not achieve clinical remission by Week 12, or |
| | experience a clinical relapse after clinical remission, may have their prednisone dose |
| | modified per the investigator but remain blinded on study treatment (secukinumab or |
| | placebo). These participants are referred to as being on Escape treatment. For |
| | participants who receive Escape treatment, GC will be administered open-label (OL) |
| | with dosing as determined by the investigator in accordance with standard of care. |
| | Alternatively, participants could discontinue study treatment to receive alternative |
| | medication(s) for GCA as Rescue treatment at the discretion of the investigator. |
| | Treatment Period 2 (Week 56 dosing to Week 108): At Week 56, participants will be |
| | classified and receive further treatment as follows: |
| | Participants with sustained clinical remission at Week 56: Participants in sustained |
| | clinical remission at Week 56 initially randomized to secukinumab, will be re- |
| | randomized 1:1 to double-blinded secukinumab 300 mg or placebo s.c. to follow a |
| | randomized withdrawal design to Week 108. Those participants who were initially |
| | randomized to placebo will have a dummy re-randomization and continue receiving |
| | placebo in a blinded manner. Re-randomization will be stratified by the participant's |
| | baseline GCA status (new-onset or relapsing GCA). As prednisone had already been |
| | discontinued at Week 26 (initial secukinumab arm) or Week 52 (initial placebo arm), |
| | these participants will not receive concomitant prednisone treatment. |
| | If a participant experiences a clinical relapse during Treatment Period 2, study |
| | treatment will be discontinued and the participant will be offered open-label (OL) |
| | secukinumab 300 mg s.c. GCs will also be given, with dosing as determined by the |
| | investigator in accordance with standard of care. |
| | Participants on Escape treatment and those not in sustained clinical remission |
| | at Week 56: Participants not in sustained clinical remission at Week 56 will be offered |
| | OL secukinumab 300 mg s.c. q4w treatment with dosing of concomitant GC as |
| | determined by the investigator, in accordance with standard of care. Participants who |
| | receive OL secukinumab but do not achieve clinical remission by 12 weeks after |
| | starting OL secukinumab, or have a clinical relapse after achieving clinical remission, |
| | will be discontinued from OL secukinumab and receive further treatment for GCA at the |
| | discretion of the investigator. Participants will attend the end of treatment (EOT) and |
| | end of study (EOS) visits. |
| | Follow-up period: Following the EOT visit at Week 108, there will be an EOS visit at |
| | Week 116 for safety assessments. |
| | Participants who are on blinded study treatment (secukinumab or placebo) at |
| | Week 108: Participants who are on blinded study treatment and in sustained clinical |
| | remission at Week 108 (i.e., are only on study treatment, without concomitant GCs), |
| | will complete treatment (last dose at Week 104), complete the EOT visit at Week 108, |
| | and EOS visit at Week 116. For participants, who in the opinion of the investigator in |
| | accordance with standard practice, will derive clinical benefit from receiving |
| | secukinumab post-trial, every effort will be made to provide OL secukinumab in |
| | accordance with the local laws and regulations. For participants who start OL |
| | secukinumab following completion of study treatment, the EOS visit at Week 116 will |
| | be waived. |
| | Participants who are on OL secukinumab at Week 108: Participants who are on OL |
| | secukinumab at Week 108 will complete treatment (last dose at Week 104), complete |
| | the EOT visit at Week 108, and EOS visit at Week 116. For participants who in the |
| | opinion of the investigator are still deriving clinical benefit from OL secukinumab, every |
| | effort will be made to continue provision of OL secukinumab in accordance with the |
| | local laws and regulations. If post-trial access to OL secukinumab is provided, the EOS |
| | visit at Week 116 will be waived. For participants who have not yet received 12 weeks |
| | of OL secukinumab, the investigator will assess if the participant is deriving clinical |
| | benefit from OL secukinumab and should continue treatment in post-trial access. |
| Study | Male or female patients at least 50 years of age with new-onset or relapsing GCA. A |
| population | minimum of 30% participants must be enrolled in each category of new-onset and |
| | relapsing patients. |
| | New-onset GCA is defined as GCA that is diagnosed within 6 weeks of Baseline. |
| | Relapsing GCA is defined as GCA diagnosed > 6 weeks before Baseline with a |
| | recurrence of active disease following the institution of appropriate treatment |
| | course per current guidelines. |
| Key Inclusion | Patients eligible for inclusion in this study must meet all of the following criteria: |
| criteria | Signed informed consent must be obtained prior to participation in the study. |
| | Patient must be able to understand and communicate with the investigator and |
| | comply with the requirements of the study. |
| | Male or non-pregnant, non-lactating female patients at least 50 years of age. |
| | Diagnosis of GCA based on meeting all of the following criteria: |
| | Age at onset of disease ≥ 50 years. |
| | Unequivocal cranial symptoms of GCA (new-onset localized headache, scalp |
| | or temporal artery tenderness, ischemia-related vision loss, or otherwise |
| | unexplained mouth or jaw pain upon mastication), and/or symptoms of |
| | polymyalgia rheumatica (PMR) (defined as shoulder and/or hip girdle pain |
| | associated with inflammatory morning stiffness) and/or symptoms of limb |
| | ischemia (claudication). |
| | Temporal artery biopsy (TAB) revealing features of GCA and/or cross- |
| | sectional imaging study such as ultrasound (e.g. cranial or axillary), MRA, |
| | CTA, or PET-CT with evidence of vasculitis. |
| | Active disease as defined by meeting both of the following within 6 weeks of |
| | Baseline: |
| | Presence of signs or symptoms of GCA |
| | Elevated erythrocyte sedimentation rate (ESR) ≥ 30 mm/hr or C-reactive |
| | protein (CRP) ≥ 10 mg/L attributed to active GCA or active GCA on TAB or |
| | imaging study |
| | Patients to meet definition of new-onset GCA or relapsing GCA: |
| | Definition of new-onset GCA: diagnosis of GCA within 6 weeks of Baseline |
| | visit |
| | Definition relapsing GCA: diagnosis of GCA > 6 weeks before Baseline |
| | visit and patient has experienced recurrence of active disease following the |
| | institution of a treatment. |
| | Patients must be eligible to receive prednisone (or equivalent) 20 mg-60 mg daily |
| | at Baseline. |
| | Patients taking MTX (≤25 mg/week) are allowed to continue their medication |
| | provided they have taken it for at least 3 months, are on a stable dose for at least |
| | 4 weeks prior to randomization, and if they are on stable folic acid treatment |
| | before randomization. |
| Key Exclusion | Previous exposure to secukinumab or other biologic drug directly targeting IL-17 |
| criteria | or IL-17 receptor. |
| | Patients treated with any cell-depleting therapies. |
| | Previous participation in clinical trial for GCA. |
| | Patients who have been treated with inhibitors directly targeting IL-1, or IL-1 |
| | receptor, IL-12 and IL-23, or abatacept within 4 weeks or within 5 half-lives of the |
| | drug (whichever is longer) prior to Baseline. |
| | Treatment with tocilizumab, other IL-6/IL6-R inhibitor or JAK inhibitor within 12 |
| | weeks or within 5 half-lives of the drug (whichever is longer) prior to Baseline, or if |
| | patient did not respond to or experienced a relapse during treatment any time |
| | before Baseline. |
| | Any treatment received for GCA other than GCs and patient did not respond to |
| | treatment or experienced a relapse during treatment any time before Baseline. |
| | Patients treated with i.v. immunoglobulins or plasmapheresis within 8 weeks prior |
| | to Baseline. |
| | Patients treated with cyclophosphamide, tacrolimus, everolimus, |
| | hydroxychloroquine, cyclosporine A, azathioprine, sulfasalazine, mycophenolate |
| | mofetil within 6 months prior to Baseline. |
| | Patients treated with leflunomide within 8 weeks of Baseline unless a |
| | cholestyramine washout has been performed in which case the patient must be |
| | treated within 4 weeks of Baseline. |
| | Patients treated with an alkylating agent within 5 years prior to Baseline, unless |
| | specified in other exclusion criteria. |
| | Patients requiring systemic chronic glucocorticoid therapy for any other reason |
| | than GCA. |
| | Receipt of > 100 mg daily intravenous methylprednisolone pulse therapy within 6 |
| | weeks prior to Baseline. |
| | Patients requiring chronic (i.e., not occasional “prn”) high potency opioid |
| | analgesics for pain management. |
| | Patients treated with any investigational agent within 4 weeks or within 5 half-lives |
| | of the drug (whichever is longer) prior to Baseline. |
| | Contraindication or hypersensitivity to secukinumab. |
| | Active ongoing inflammatory diseases other than GCA that might confound the |
| | evaluation of the benefit of secukinumab therapy, including inflammatory bowel |
| | disease or uveitis. |
| | Major ischemic event (e.g. myocardial infarction, stroke, etc.) or transient |
| | ischemic attack (TIA) (except ischemia-related vision loss), related or unrelated to |
| | GCA, within 12 weeks of screening. |
| | Confirmed diagnosis of any primary form of systemic vasculitis, other than GCA. |
| | Any other biologics within 4 weeks or within 5 half-lives of the drug (whichever is |
| | longer) prior to Baseline. |
| | Active ongoing diseases which in the opinion of the |
| | investigator immunocompromises the patient and/or places the patient at |
| | unacceptable risk for treatment with immunomodulatory therapy. |
| | Active systemic infections during the last 2 weeks (exception: common cold) prior |
| | to randomization. |
| | History of ongoing, chronic or recurrent infectious disease or evidence of |
| | tuberculosis infection as defined by a positive QuantiFERON TB- Gold Plus test. |
| | Patients with a positive test may participate in the study if further work up |
| | (according to local practice/guidelines) establishes conclusively that the patient |
| | has no evidence of active tuberculosis. If presence of latent tuberculosis |
| | is established then treatment according to local country guidelines must be |
| | initiated prior to randomization. |
| | Live vaccinations within 6 weeks prior to Baseline or planned vaccination during |
| | study participation until 12 weeks after last study treatment administration. |
| Study | Treatment Period 1 (Baseline (BSL) to Week 56 pre-dose): |
| treatment | At Baseline, all eligible participants will be randomized to one of the two treatment arms |
| | in a 2:1 ratio: |
| | Group 1: Secukinumab 300 mg (2 mL PFS) s.c. at BSL, Weeks 1, 2, 3, followed |
| | by administration every four weeks starting at Week 4. Secukinumab will be given |
| | in combination with a protocol-specified 26-week prednisone taper regimen |
| | Group 2: Placebo to secukinumab 300 mg (2 mL PFS) s.c. at BSL, Weeks 1, 2, 3, |
| | followed by administration every four weeks starting at Week 4. Placebo will be |
| | given in combination with a protocol-specified 52-week prednisone taper regimen. |
| | Randomization in the two groups will be stratified by the participant's GCA status (new- |
| | onset or relapsing GCA) and baseline prednisone dose (≤30 mg/day or >30 mg/day |
| | prednisone). |
| | Treatment Period 2 (Week 56 dosing to Week 108): |
| | Participants with sustained clinical remission at Week 56 who were initially |
| | randomized to secukinumab will be re-randomized 1:1 (stratified by the participant's |
| | baseline GCA status of new-onset or relapsing GCA) to follow a randomized withdrawal |
| | design to Week 104: |
| | Group 1: Secukinumab 300 mg (2 mL PFS) s.c. at every four weeks starting at |
| | Week 56. |
| | Group 2: Placebo to secukinumab 300 mg (2 mL PFS) s.c. at every four weeks |
| | starting at Week 56. |
| | Those participants who were initially randomized to Group 2 (placebo) will have a |
| | dummy re-randomization and continue receiving placebo in a blinded manner. |
| Treatment of | Secukinumab 300 mg s.c. + prednisone 26-week taper regimen vs. placebo + |
| interest | prednisone 52-week taper regimen |
| Efficacy | GCA assessment (signs and symptoms) |
| assessments | Inflammatory markers |
| | ESR |
| | CRP |
| | Cumulative glucocorticoid dosing |
| Key safety | Physical examination |
| assessments | Vital signs |
| | Laboratory evaluations |
| | Glucocorticoid toxicity index (GTI) |
| | Adverse events (AEs)/serious adverse events (SAEs) |
| Other | Physicians global assessment of disease activity |
| assessments | Patient reported outcomes (PRO) |
| | EQ-5D |
| | SF-36 |
| | FACIT-Fatigue |
| Data analysis | The primary efficacy analysis is to show subcutaneous secukinumab 300 mg in |
| | combination with a 26-week prednisone taper regimen is superior to placebo in |
| | combination with a 52-week prednisone taper regimen. The following null and |
| | alternative hypothesis will be tested at one-sided 2.5% level of significance to address |
| | the primary efficacy objective for sustained remission at Week 52: |
| | H₀₁: p₁ − p₀ <= 0 vs. Hₐ₁: p₁ − p₀ > 0 |
| | where p₁ is the sustained remission rate for secukinumab + 26-week prednisone taper |
| | group and p₀ is the sustained remission rate for placebo + 52-week prednisone taper |
| | group. |
| | In other words: |
| | H₀₁: secukinumab 300 mg + 26-week prednisone taper is not superior to placebo + 52- |
| | week prednisone taper with respect to sustained remission at Week 52 |
| | Hₐ₁: secukinumab 300 mg + 26-week prednisone taper is superior to placebo + 52- |
| | week prednisone taper with respect to sustained remission at Week 52 |
| | Difference in response proportion will be computed for comparisons of secukinumab |
| | group vs. placebo group using a logistic regression adjusting baseline prednisone dose |
| | (≤30 mg/day, >30 mg/day) and new-onset/relapsing GCA. Difference in marginal |
| | response proportions with p-value and respective 95% confidence interval will be |
| | estimated from the logistic regression model. |
| | The primary analysis will be based on the full analysis set (FAS). |
| | Safety analyses will include summaries of AEs, laboratory measurements, and vital |
| | signs. Full details of all data analyses will be specified in statistical analysis plan (SAP). |
| Key words | Giant cell arteritis (GCA), secukinumab, monoclonal antibody, AIN457, prednisone |
| | taper regimen, glucocorticoid (GC) |

### 1.2 Purpose

The purpose of this study is to demonstrate the efficacy and safety of secukinumab 300 mg administered subcutaneously (s.c.) for 52 weeks in combination with prednisone tapered over 26 weeks in adult participants with newly diagnosed or relapsing giant cell arteritis (GCA).

### 2 Objectives and Endpoints

TABLE 0-2
Objectives and related endpoints

| Objective(s) | Endpoint(s) |
|---|---|
| Primary objective(s) | Endpoint(s) for primary objective(s) |
| The primary objective is to demonstrate | Primary endpoint: Sustained remission |
| that the efficacy of secukinumab 300 mg | at Week 52. |
| s.c. in combination with a 26-week GC | Sustained remission at Week 52 is defined |
| taper regimen is superior to placebo in | as participant meeting all of the following: |
| combination with a 52-week GC taper | |
| regimen in participants with GCA based on | achieved remission at Week 12 |
| sustained remission at Week 52 | AND all of the following, sustained from |
| | Week 12 to Week 52 |
| | no recurrence of signs or symptoms, |
| | attributable to GCA, that require an |
| | increase in prednisone dose |
| | compared to the pre-specified taper |
| | or rescue treatment |
| | no elevation of ESR to ≥ 30 mm/hr, |
| | attributable to GCA, that requires an |
| | increase in prednisone dose |
| | compared to the pre-specified taper |
| | or rescue treatment |
| | normalization of CRP (<10 mg/L) |
| | without elevation to ≥10 mg/L at 2 |
| | consecutive scheduled visits |
| | no non-adherence to the pre- |
| | specified prednisone taper by an |
| | increase of dose for GCA. |
| | Remission at Week 12 is defined as |
| | participant meeting all of the following at |
| | Week 12 and having had no recurrence of |
| | the following if reached remission prior to |
| | Week 12: |
| | no signs or symptoms attributable to |
| | GCA, that require an increase in |
| | prednisone dose compared to the |
| | pre-specified taper or rescue |
| | treatment. |
| | no elevation of ESR to ≥ 30 mm/hr |
| | attributable to GCA, that requires an |
| | increase in prednisone dose |
| | compared to the pre-specified taper |
| | or rescue treatment. |
| | no elevation of CRP (≥10 mg/L) |
| | at 2 consecutive scheduled visits. |
| | no non-adherence to the pre- |
| | specified prednisone taper by an |
| | increase of dose for GCA. |
| Secondary objective(s) | Endpoint(s) for secondary objective(s) |
| To assess the efficacy of secukinumab in | Time to clinical failure as measured in |
| combination with a 26-week GC taper | days through Week 52 |
| regimen compared to placebo in | Clinical failure is defined as first time |
| combination with a 52-week GC taper | meeting one of the following: |
| regimen in participants with GCA based | remission not achieved by Week 12 |
| on time to clinical failure through Week | GCA clinical relapse after achieving |
| 52 | remission |
| | GCA clinical relapse is defined as meeting |
| | at least one of the following: |
| | recurrence of signs or |
| | symptoms attributable to GCA, |
| | that require an increase in |
| | prednisone dose compared to |
| | the pre-specified prednisone |
| | taper or rescue treatment |
| | elevated ESR ≥ 30 mm/hr, |
| | attributable to GCA, that |
| | requires an increase in |
| | prednisone dose compared to |
| | the pre-specified taper or rescue |
| | treatment |
| | non-adherence to the pre- |
| | specified prednisone taper by |
| | an increase of dose for GCA. |
| | study treatment discontinuation. |
| To assess the efficacy of secukinumab in | Cumulative GC dose through Week 52 |
| combination with a 26-week GC taper | |
| regimen compared to placebo in | |
| combination with a 52-week GC taper | |
| regimen, in participants with GCA, based | |
| on cumulative GC dose through Week 52 | |
| To assess the effect on participant's QoL of | Change in SF-36 score (PCS) |
| secukinumab in combination with a 26- | |
| week GC taper regimen compared to | |
| placebo in combination with a 52-week GC | |
| taper regimen, in participants with GCA | |
| based on change of SF-36 score (PCS) at | |
| Week 52 | |
| To assess the effect of secukinumab in | Change in Glucocorticoid Toxicity |
| combination with a 26-week GC taper | Index (GTI) as measured by the |
| regimen compared to placebo in | Aggregate Improvement Score (AIS) at |
| combination with a 52-week GC taper | Week 52 |
| regimen, in participants with GCA based on | |
| change in Glucocorticoid Toxicity Index | |
| (GTI) at Week 52 | |
| To assess the effect on participant's QoL of | Change in FACIT-Fatigue score |
| secukinumab in combination with a 26- | |
| week GC taper regimen compared to | |
| placebo in combination with a 52-week GC | |
| taper regimen, in participants with GCA | |
| based on change in FACIT-Fatigue score | |
| at Week 52 | |
| To assess the effect on participant's QoL of | Change in EQ-5D score |
| secukinumab treatment in combination with | |
| a 26-week GC taper regimen compared to | |
| placebo in combination with a 52-week GC | |
| taper regimen, in participants with GCA | |
| based on change in EQ-5D | |
| score at Week 52 | |
| To evaluate the safety and tolerability of | Safety and tolerability demonstrated by |
| secukinumab | assessing: |
| | 1. Adverse events (AEs) and serious |
| | adverse events (SAEs) (incidence, |
| | severity, and relationship to study |
| | drug) |
| | 2. Clinically significant changes in |
| | clinical laboratory measures and |
| | vital signs, as assessed by the |
| | Investigator |

### Primary Estimands

The primary scientific question of interest is: what is the relative effect of secukinumab 300 mg s.c. in combination with a 26-week prednisone taper regimen compared to placebo in combination with a 52-week prednisone taper regimen in participants with GCA on proportion of participants achieving sustained remission at Week 52, without study treatment discontinuation and without escape medication or rescue medication.

The primary estimand is characterized by the following attributes:
Population: defined through appropriate inclusion/exclusion criteria to reflect the targeted GCA population.
Treatment of interest: Secukinumab 300 mg s.c.+prednisone 26-week taper regimen vs. placebo+prednisone 52-week taper regimen

### Primary Variable:

Sustained remission status at Week 52 is defined as achieved remission at Week 12 and maintained remission from Week 12 through Week 52, without study treatment discontinuation and without escape medication or rescue medication for GCA.

### Handling of Remaining Intercurrent Events:

Study treatment discontinuation due to any reason: non-responder (composite strategy)
Prohibited medication: Medications that are not allowed by the protocol and that have a potential confounding effect on the efficacy of secukinumab. Participants who receive prohibited medication and discontinue study treatment will be considered as non-responder (as defined in the previous intercurrent event-study treatment discontinuation) (composite strategy). If the participant is not discontinued from study treatment after taking a prohibited medication, the potential confounding effects due to intake of prohibited medication are ignorable (treatment policy strategy).
Escape treatment for GCA: Receipt of Escape treatment including GC at a dose determined by the investigator in accordance with standard of care is captured as a component of the primary variable. Participants who receive Escape treatment will be considered as non-responders (composite strategy).
Rescue treatment for GCA: Receipt of Rescue treatment is captured as a component of the primary variable. In addition, participants who receive Rescue treatment would also be discontinued from study treatment. These participants are considered as non-responders (composite strategy).

### Summary Measure:

Difference in sustained remission rate between secukinumab group and placebo group.

### Secondary Estimands

For all secondary endpoints, supplementary estimands of interest may be specified in statistical analysis plan.

### Time to Clinical Failure

The scientific question of interest: what is the relative effect of secukinumab 300 mg s.c. in combination with a 26-week prednisone taper regimen compared to placebo in combination with a 52-week prednisone taper regimen, in prolonging time to clinical failure (considering Escape/Rescue treatment for GCA, study treatment discontinuation and study discontinuation as clinical failure) in patients with GCA.

The estimand is characterized by the following attributes:
Population: Defined through appropriate inclusion/exclusion criteria to reflect the targeted GCA population.
Treatment of interest: Secukinumab 300 mg s.c.+prednisone 26-week taper regimen vs. placebo+prednisone 52-week taper regimen.
Primary Variable: Time to clinical failure as described herein, considering study treatment discontinuation and Escape/Rescue treatment for GCA as clinical failure.

### Handling of Remaining Intercurrent Events:

Study treatment discontinuation before Week 52 due to any reason: composite strategy (reflected in Variable above).
Escape treatment for GCA: composite strategy (reflected in Variable above).
Rescue treatment for GCA: composite strategy (reflected in Variable above).
Study discontinuation before Week 52 due to any reason: composite strategy (reflected in Variable above).
Summary measure: hazard ratio for time to clinical failure between secukinumab group and placebo group.

### Cumulative GC Dose Through Week 52

The scientific question of interest: what is the relative effect of secukinumab 300 mg s.c. in combination with a 26-week prednisone taper regimen compared to placebo in combination with a 52-week prednisone taper regimen in participants with GCA on the cumulative GC dose through Week 52, had patients continued on study treatment without Rescue treatment for GCA.

### The Estimand is Characterized by the Following Attributes:

Population: Defined through appropriate inclusion/exclusion criteria to reflect the targeted GCA population.
Treatment of interest: Secukinumab 300 mg s.c.+prednisone 26-week taper regimen vs. placebo+prednisone 52-week taper regimen.
Variable: Cumulative GC dose through Week 52.

### Handling of Remaining Intercurrent Events:

Use of prohibited medication before Week 52 due to any reason will be handled via hypothetical strategy (had prohibited medications not been available to participants).
Summary measure: Difference in average (median) cumulative GC dose between secukinumab group and placebo group.

### Pros (SF-36 PCS, EQ-5D, FACIT-Fatigue Score) and GTI Total Score

The scientific question of interest: what is the relative effect of secukinumab 300 mg s.c. in combination with a 26-week prednisone taper regimen compared to placebo in combination with a 52-week prednisone taper regimen in participants with GCA on the change in PRO score and GTI score from baseline to Week 52, had patients continued on study treatment without Rescue treatment for GCA.
Population: Defined through appropriate inclusion/exclusion criteria to reflect the targeted GCA population.
Treatment of interest: Secukinumab 300 mg s.c.+prednisone 26-week taper regimen vs. placebo+prednisone 52-week taper regimen.
Variable (SF-36 PCS, EQ-5D, FACIT-Fatigue score, GTI total score): Change in the variable of interest from Baseline to Week 52.

### Handling of Remaining Intercurrent Events:

Study treatment discontinuation before Week 52 due to any reason: Had a participant not discontinued (hypothetical)
Rescue treatment for GCA: Had rescue treatment not been available to participants (hypothetical).
Study discontinuation before Week 52 due to any reason: Had a participant not discontinued (hypothetical).
Summary measure: Difference in means between secukinumab group and placebo group.

### Study Design

This randomized, parallel-group, double-blind, placebo-controlled, multicenter, Phase III study is designed to evaluate the efficacy of secukinumab in combination with a 26-week prednisone taper regimen compared to placebo in combination with a 52-week prednisone taper regimen based on sustained remission at Week 52 in participants with newly diagnosed or relapsing GCA. Approximately 240 participants will be enrolled in the study.

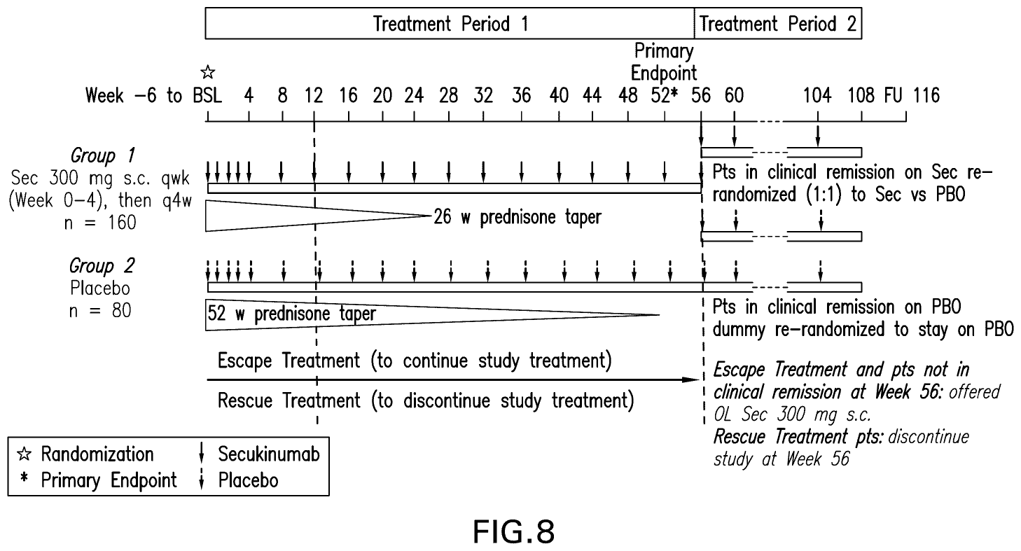

The study will consist of 6-week screening period, a 56-week treatment period (Treatment Period 1), a second treatment period of 52 weeks (Treatment Period 2) and an 8-week safety Follow-Up Period (FIG. 8).
Screening period: During the screening period, it is expected that participants will be treated for GCA with GCs as per the investigator's judgement. By the end of the screening period participants should be able to switch to the sponsor-provided prednisone at Baseline in order to follow the protocol-defined prednisone tapering regimens. Starting prednisone doses at Baseline are 20, 25, 30, 35, 40, 50 or 60 mg/day at the investigator's discretion.

In the event of a major healthcare disruption (e.g., a pandemic or epidemic) that limits or prevents on-site visits to the study sites the Screening Period may be extended to a maximum of 8 weeks before Baseline.

Treatment Period 1 (Baseline (BSL) to Week 56 pre-dose): At Baseline, participants will be randomized to one of the following 2 arms in a 2:1 ratio:
Group 1: Secukinumab 300 mg (2 mL PFS) s.c. at BSL, Weeks 1, 2, 3, followed by administration every four weeks starting at Week 4. Secukinumab will be given in combination with a specified 26-week prednisone taper regimen
Group 2: Placebo to secukinumab 300 mg (2 mL PFS) s.c. at BSL, Weeks 1, 2, 3, followed by administration every four weeks starting at Week 4. Placebo will be given in combination with a specified 52-week prednisone taper regimen.

In order to achieve a balanced distribution of baseline prednisone dose and GCA status in each treatment arm, randomization in the two groups will be stratified by the participant's GCA status (new-onset or relapsing GCA) and baseline prednisone dose (≤30 mg/day or >30 mg/day prednisone).

Participants who do not achieve clinical remission by Week 12, or experience a clinical relapse after clinical remission, may have their GC dose modified as determined by the investigator in accordance with standard of care but remain on blinded study treatment (secukinumab or placebo). These participants are referred to as being on Escape treatment. For participants who receive Escape treatment, GC will be administered open-label with dosing as determined by the investigator in accordance with standard of care.

Clinical remission at Week 12 is defined as participant meeting all of the following at Week 12 and having had no recurrence of the following if reached remission prior to Week 12:
no signs or symptoms attributable to GCA, that require an increase in prednisone dose compared to the pre-specified taper (or rescue treatment)
no elevation of ESR to ≥30 mm/hr, attributable to GCA, that requires an increase in prednisone dose compared to the pre-specified taper (or rescue treatment). no non-adherence to the pre-specified prednisone taper by an increase of dose for GC

As CRP lab values are blinded, CRP values are not part of the clinical remission definition.

For participants who receive Escape treatment, prednisone will be administered open-label. Participants on Escape treatment remain in the study and should continue to attend all subsequent scheduled visits.

If participants receive Rescue treatment (another treatment for GCA and not only prednisone), then study treatment will be discontinued before Rescue treatment is started. Such participants may remain in the study and attend all subsequent scheduled visit assessments (in Treatment Period 1 and a follow-up visit).
Treatment Period 2 (Week 56 dosing to Week 108): At Week 56, participants will be classified and receive further treatment as follows:
Participants with sustained clinical remission at Week 56: Participants who are in sustained clinical remission (as defined above) at Week 56, who were initially randomized to secukinumab will be re-randomized 1:1 to double-blinded secukinumab 300 mg or placebo s.c. to follow a randomized withdrawal design to Week 108. Those participants who were initially randomized to placebo will have a dummy re-randomization and continue receiving placebo in a blinded manner. Re-randomization will be stratified by the participant's baseline GCA status (new-onset or relapsing GCA). As prednisone had already been discontinued at Week 26 (initial secukinumab arm) or Week 52 (initial placebo arm), these participants will not receive concomitant prednisone treatment.

If a participant experiences a clinical relapse during Treatment Period 2, study treatment will be discontinued and the participant will be offered open-label (OL) secukinumab 300 mg s.c. Treatment initiation of OL secukinumab will be similar to that for initial secukinumab treatment, and administered every week (qw) for the first 4 weeks followed by administration every 4 weeks (q4w) starting 4 weeks from the initiation of OL secukinumab. GCs will also be given, with dosing determined by the investigator in accordance with standard of care.

Participants who do not achieve clinical remission by 12 weeks after starting OL secukinumab or have a clinical relapse on OL secukinumab during Treatment Period 2, will discontinue OL secukinumab and receive further treatment for GCA at the discretion of the investigator. Participants will attend the end of treatment (EOT) and end of study (EOS) visits.
Participants not in sustained clinical remission at Week 56: Participants who are not in sustained clinical remission at Week 56 including those who are on Escape treatment will be offered OL secukinumab 300 mg s.cq4w treatment with dosing of concomitant GC as determined by the investigator in accordance with standard of care. Treatment initiation of OL secukinumab will be similar to that for initial secukinumab treatment, and administered every week (qw) for the first 4 weeks followed by administration every 4 weeks (q4w) starting 4 weeks from the initiation of OL secukinumab. Participants who receive OL secukinumab and do not achieve clinical remission by 12 weeks after starting OL secukinumab or have a clinical relapse after achieving clinical remission, will discontinue study treatment and attend the EOT and EOS visits. Further treatment for GCA will be determined by the investigator.
Follow-up period: Following the EOT visit at Week 108, there will be an EOS visit at Week 116 for safety assessments.
Participants who are on blinded study treatment (secukinumab or placebo) at Week 108: Participants who are on blinded study treatment and in sustained clinical remission at Week 108 (i.e., are only on study treatment, without concomitant GCs), will complete treatment (last dose at Week 104), complete the EOT visit at Week 108, and EOS visit at Week 116. For participants, who in the opinion of the investigator in accordance with standard practice, will derive clinical benefit from receiving secukinumab post-trial, every effort will be made to provide OL secukinumab in accordance with the local laws and regulations. For participants who start OL secukinumab following completion of study treatment, the EOS visit at Week 116 will be waived.
Participants who are on OL secukinumab at Week 108: Participants who are on OL secukinumab at Week 108 will complete treatment (last dose at Week 104), complete the EOT visit at Week 108, and EOS visit at Week 116. For participants who in the opinion of the investigator are still deriving clinical benefit from OL secukinumab, every effort will be made to continue provision of OL secukinumab in accordance with the local laws and regulations. If post-trial access to OL secukinumab is provided, the EOS visit at Week 116 will be waived. For participants who have not yet received 12 weeks of OL secukinumab, the investigator will assess if the participant is deriving clinical benefit from OL secukinumab and should continue treatment in post-trial access.

### Imaging Sub-Study

MRA is a non-invasive radiology technique that has no X-ray radiation exposure. However, the MRA scanning equipment may cause a feeling of claustrophobia in susceptible persons and the presence of metal in the body may also be a safety hazard or affect the MRA image quality. Therefore, participants can only be considered for this type of imaging if they do not have any contraindications to MRA. For more information, see Exclusion Criteria.

The MRA measurement will require the injection of a gadolinium-based contrast agent (GBCA) during each MRA session. There is recent evidence of gadolinium deposition in brain tissues following use of GBCAs. Although no symptoms or diseases linked to gadolinium accumulation in the brain have been identified, health authorities have taken a precautionary approach (e.g., GBCA EMA restriction re brain deposit), noting that data on the long-term effects in the brain are limited. This led to the suspension of several linear GBCAs and the recommendation that another class of GBCAs, known as macrocyclic agents, are to be used as they are deemed more stable and have a lower propensity to release gadolinium than linear agents. Although this is highly debated, the current view is that such agents, especially again the linear gadolinium agents, may also increase the risk of a rare but serious disease called nephrogenic systemic fibrosis (NSF). In support of this, a recent review of 639 patients with biopsy-confirmed NFS showed that the rate of NFS cases per million exposures is 1.52 for group I (i.e., linear) GPCAs, however only 0.008 for group II (i.e., macrocyclic) agents (Attari et al 2019). To further reduce this risk and in accordance with health authority guidance (e.g., GBCA UK health authority guidance regarding NSF, GBCA FDA guidance regarding NSF), people with severe kidney failure, participants with previous severe allergic/anaphylactoid reaction to a gadolinium-based contrast agent, and participants with severe renal disease [estimated glomerular filtration rate (eGFR)<30 mL/min/1.73 m²] or acutely deteriorating renal function, will be excluded from participating in the imaging sub-study as stipulated in the Exclusion Criteria.

### Inclusion Criteria

Patients eligible for inclusion in this study must meet all of the following criteria:
1. Signed informed consent must be obtained prior to participation in the study.
2. Patient must be able to understand and communicate with the investigator and comply with the requirements of the study.
3. Male or non-pregnant, non-lactating female patients at least 50 years of age.
4. Diagnosis of GCA based on meeting all of the following criteria:
   Age at onset of disease ≥50 years.
   Unequivocal cranial symptoms of GCA (new-onset localized headache, scalp or 1 mouth or jaw pain upon mastication), and/or symptoms of polymyalgia rheumatica (PMR) (defined as shoulder and/or hip girdle pain associated with inflammatory morning stiffness) and/or symptoms of limb ischemia (claudication).
   TAB revealing features of GCA and/or cross-sectional imaging study such as ultrasound (e.g. cranial or axillary), MRA, CTA, or PET-CT with evidence of vasculitis.
5. Active disease as defined by meeting both of the following within 6 weeks of Baseline:
   Presence of signs or symptoms of GCA
   Elevated ESR≥30 mm/hr or CRP≥10 mg/L attributed to active GCA or active GCA on Temporal Artery Biopsy (TAB) or imaging study.
6. Patients to meet definition of new-onset GCA or relapsing GCA:
   Definition of new-onset GCA: diagnosis of GCA within 6 weeks of Baseline visit
   Definition relapsing GCA: diagnosis of GCA>6 weeks before Baseline visit and patient has experienced recurrence of active disease following the institution of a treatment.
7. Patients must be eligible to receive prednisone (or equivalent) 20 mg-60 mg daily at Baseline.
8. Patients taking MTX (≤25 mg/week) are allowed to continue their medication provided they have taken it for at least 3 months and are on a stable dose for at least 4 weeks prior to randomization and if they are on stable folic acid treatment before randomization.

### Exclusion Criteria

Patients meeting any of the following criteria are not eligible for inclusion in this study. No additional exclusions may be applied by the investigator, in order to ensure that the study population will be representative of all eligible patients.
1. Pregnant or nursing (lactating) women where pregnancy is defined as the state of a female after conception and until the termination of gestation, confirmed by a positive human chorionic gonadotropin (hCG) laboratory test.
2. Women of childbearing potential, defined as all women physiologically capable of becoming pregnant, unless they are using effective methods of contraception during dosing of study treatment and for a minimum of 20 weeks after the last dose of secukinumab. Basic contraception methods include:
   Total abstinence (when this is in line with the preferred and usual lifestyle of the patient). Periodic abstinence (e.g., calendar, ovulation, symptothermal, post-ovulation methods) and withdrawal are not acceptable methods of contraception.
   Female sterilization (have had surgical bilateral oophorectomy with or without hysterectomy), total hysterectomy or tubal ligation at least 6 weeks before taking investigational drug. In case of oophorectomy alone, only when the reproductive status of the woman has been confirmed by follow-up hormone level assessment.
   Male sterilization (at least 6 months prior to screening). For female patients on the study, the vasectomized male partner should be the sole partner for that patient
   Barrier methods of contraception: Condom or Occlusive cap (diaphragm or cervical/vault caps).
   Use of oral, (estrogen and progesterone), injected or implanted hormonal methods of contraception or other forms of hormonal contraception that have comparable efficacy. In case of use of oral contraception women should have been stable on the same pill for a minimum of 3 months before taking study treatment.
   Women are considered post-menopausal and not of childbearing potential if they have had at least 12 months of natural (spontaneous) amenorrhea with an appropriate clinical profile (e.g., age appropriate, history of vasomotor symptoms) or have had surgical bilateral oophorectomy (with or without hysterectomy), total hysterectomy or tubal ligation at least 6 weeks ago. In the case of oophorectomy alone, only when the reproductive status of the woman has been confirmed by follow-up hormone level assessment is she considered not of childbearing potential.

If local regulations deviate from the contraception methods listed above to prevent pregnancy, local regulations apply and will be described in the Informed Consent Form (ICF).
3. Previous exposure to secukinumab or other biologic drug directly targeting IL-17 or IL-17 receptor.
4. Patients treated with any cell-depleting therapies.
5. Previous participation in clinical trial for GCA.
6. Patients who have been treated with inhibitors directly targeting IL-1, or IL-1 receptor, IL-12 and IL-23, or abatacept within 4 weeks or within 5 half-lives of the drug (whichever is longer) prior to Baseline.
7. Treatment with tocilizumab, other IL-6/IL6-R inhibitor or JAK inhibitor within 12 weeks or within 5 half-lives of the drug (whichever is longer) prior to Baseline, or if patient did not respond to or experienced a relapse during treatment any time before Baseline.
8. Any treatment received for GCA other than GCs and patient did not respond to treatment or experienced a relapse during treatment any time before Baseline.
9. Patients treated with i.v. immunoglobulins or plasmapheresis within 8 weeks prior to Baseline.
10. Patients treated with cyclophosphamide, tacrolimus, everolimus, hydroxychloroquine, cyclosporine A, azathioprine, sulfasalazine, mycophenolate mofetil within 6 months prior to Baseline.
11. Patients treated with leflunomide within 8 weeks of Baseline unless a cholestyramine washout has been performed in which case the patient must be treated within 4 weeks of Baseline.
12. Patients treated with an alkylating agent within 5 years prior to Baseline, unless specified in other exclusion criteria.
13. Patients requiring systemic chronic glucocorticoid therapy for any other reason than GCA.
14. Receipt of >100 mg daily intravenous methylprednisolone pulse therapy within 6 weeks prior to Baseline.
15. Patients requiring chronic (i.e., not occasional “prn”) high potency opioid analgesics for pain management.
16. Patients treated with any investigational agent within 4 weeks or within 5 half-lives of the drug (whichever is longer) prior to Baseline.
17. Contraindication or hypersensitivity to secukinumab.
18. Active ongoing inflammatory diseases other than GCA that might confound the evaluation of the benefit of secukinumab therapy, including inflammatory bowel disease or uveitis.
19. Major ischemic event (e.g. myocardial infarction, stroke, etc.) or transient ischemic attack (TIA) (except ischemia-related vision loss), related or unrelated to GCA, within 12 weeks of screening.
20. Confirmed diagnosis of any primary form of systemic vasculitis, other than GCA.
21. Any other biologics within 4 weeks or within 5 half-lives of the drug (whichever is longer) prior to Baseline.
22. Active ongoing diseases which in the opinion of the investigator immunocompromises the patient and/or places the patient at unacceptable risk for treatment with immunomodulatory therapy.
23. Significant medical problems or diseases, including but not limited to the following: uncontrolled hypertension (≥160/95 mmHg), congestive heart failure (New York Heart Association (NYHA) status of class III or IV) and uncontrolled diabetes mellitus.
24. History of clinically significant liver disease or liver injury as indicated by abnormal liver function tests (LFTs) such as Aspartate Aminotransferase (AST), alanine aminotransferase (ALT), alkaline phosphatase (ALP) or serum bilirubin. The investigator should be guided by the following criteria:
   SGOT (AST) and SGPT (ALT) may not exceed 3×the upper limit of normal (ULN). A single parameter elevated up to and including 3×ULN should be re-checked once more as soon as possible, and in all cases, at least prior to randomization, to rule-out laboratory error.
   Alkaline phosphatase may not exceed 2×ULN. An elevation up to and including 2× ULN should be re-checked once more as soon as possible, and in all cases, at least prior to randomization, to rule-out laboratory error.
   Total bilirubin may not exceed 2×ULN. If the total bilirubin concentration is increased above 2×ULN, total bilirubin should be differentiated into the direct and indirect reacting bilirubin.
25. Patients with severely reduced kidney function [estimated glomerular filtration rate (eGFR)<30 mL/min/1.73 m² by Chronic Kidney Disease Epidemiology Collaboration (CKD-EPI)].
26. Screening total WBC count <3,000/μL, or platelets <100,000/μL or neutrophils <1,500/μL or Hgb<8.3 g/dL (83 g/L).
27. Active systemic infections during the last 2 weeks (exception: common cold) prior to randomization.
28. History of ongoing, chronic or recurrent infectious disease or evidence of tuberculosis infection as defined by a positive QuantiFERON TB-Gold Plus test. Patients with a positive test may participate in the study if further work up (according to local practice/guidelines) establishes conclusively that the patient has no evidence of active tuberculosis. If presence of latent tuberculosis is established then treatment according to local country guidelines must be initiated prior to randomization.
29. Known infection with human immunodeficiency virus (HIV), hepatitis B or hepatitis C at screening or randomization.
30. History of lymphoproliferative disease or any known malignancy or history of malignancy of any organ system within the past 5 years (except for basal cell carcinoma or actinic keratosis that have been treated with no evidence of recurrence in the past 3 months, carcinoma in situ of the cervix or non-invasive malignant colon polyps that have been removed).
31. Live vaccinations within 6 weeks prior to Baseline or planned vaccination during study participation until 12 weeks after last study treatment administration.
32. Current severe progressive or uncontrolled disease, which in the judgment of the clinical investigator renders the patient unsuitable for the trial.
33. Any medical or psychiatric condition, which, in the investigator's opinion, would preclude the patient from adhering to the protocol or completing the study per protocol.
34. Donation or loss of 400 mL or more of blood within 8 weeks before randomization.
35. History or evidence of ongoing alcohol or drug abuse, within the last 6 months before randomization.
36. Specific for MRA imaging sub-study: absolute contraindications to MRA (e.g., metallic implants, metallic foreign bodies, pacemaker, defibrillator) and to the use of gadolinium-based agents (e.g., people with severe kidney failure, patients with previous severe allergic/anaphylactoid reaction to a gadolinium-based contrast agent); patients with severe renal disease [eGFR<30 mL/min/1.73 m² by Chronic Kidney Disease Epidemiology Collaboration (CKD-EPI)], or acutely deteriorating renal function, who would be at risk of nephrogenic systemic fibrosis.

### Treatment

### Study Treatment

### Investigational and Control Drugs

Each study site will be supplied by Novartis with investigational treatment in packaging of identical appearance. The investigational treatment packaging has a 2-part label. A unique Randomization number is printed on each part of this label, which corresponds to placebo or active treatment.

An overview of study treatment administration is presented in FIG. 8. Participants will be given all secukinumab and placebo injections by the site staff on site during Treatment Period 1 (Baseline through Week 56 pre-dose). The Week 56 dose will also be given on site.

For dosing during Treatment Period 2 (Week 56 dosing through Week 108), participants will have the option of home administration starting with Week 60, after being adequately trained on the procedure. However if participants want to come to the site for Investigational Medicinal Product administration, they may do so. At Week 56, site staff will administer study treatment at the site and dispense Investigational Medicinal Product the next two months of study treatment for home administration. All participants will have an on-site visit every 2-3 months from Week 56 until the end of the study.

The PFSs are packed in a double-blind fashion and do not need to be prepared. The study treatments will be labeled as follows
Double-blind secukinumab and placebo prefilled syringes (PFSs) labeled “AIN457 300 mg/2 mL/Placebo”

Participants will also receive prednisone in open-label fashion ≥20 mg/day and double-blind fashion <20 mg/day for tapered dosing regimen from Baseline to Week 52. Participants will be given prednisone by the site staff on site at study site visits and will be supplied with sufficient prednisone for home administration between study visits.

TABLE 0-3
Investigational and control drug

| Investigational/ | | | | |
|---|---|---|---|---|
| Control Drug | | | | |
| (Name and | Pharmaceutical | Route of | | Sponsor (global |
| Strength) | Dosage Form | Administration | Supply Type | or local) |
| Secukinumab | 2 mL PFS | Subcutaneous | Double-blind | Novartis |
| 300 mg | | | Supply | |
| Secukinumab | 2 mL PFS | Subcutaneous | Double-blind | Novartis |
| Placebo | | | Supply | |
| Prednisone | 1 mg, 2.5 mg, | Oral | Open- | Novartis |
| (taper | 5 mg, 20 mg | administration | label/Double- | |
| regimen) from a | tablets in | | blind Supply | |
| dose of 20 | capsule | | | |
| mg/day to 60 | | | | |
| mg/day at | | | | |
| Baseline to 0 | | | | |
| mg/day at the | | | | |
| end of the taper | | | | |
| (open-label ≥ 20 | | | | |
| mg/day | | | | |
| and double- | | | | |
| blind < 20 | | | | |
| mg/day) | | | | |

### Additional Study Treatments

Prednisone from Baseline

Starting at Baseline, prednisone will be provided to the participant, as part of the GC taper regimen. Over-encapsulated prednisone tablets of varying strengths (1 mg, 2.5 mg, 5 mg, 20 mg) and placebo tablets will be combined in blister packs (4 blisters for each day) to fulfill the daily dosing requirement in accordance with the specified taper for the treatment group (secukinumab or placebo) to which the participant is randomized. Over-encapsulation allows for blinding of the daily prednisone dose which is required starting at prednisone doses <20 mg/day. Although also over-encapsulated tablets will be combined into blister packs for total daily dosages of ≥20 mg/day, the dose will be known to the investigator and participant.

### Treatment Arms/Group

Approximately 240 participants will be randomized to one of the following 2 treatment arms in a 2:1 (secukinumab: placebo) ratio.
Group 1: secukinumab 300 mg s.c. (2 mL)+26-week prednisone taper regimen
Group 2: placebo s.c. (2 mL)+52-week prednisone taper regimen
Stratification: In order to achieve a balanced baseline prednisone dose distribution in each treatment arm, randomization will be stratified at Baseline by the participant's GCA status (new-onset or relapsing GCA) and the baseline prednisone dose (>30 mg/day and ≤30 mg/day prednisone).
Treatment Period 1 (Baseline to Week 56 pre-dose): participants will receive secukinumab or placebo at Baseline, Week 1, 2, 3 followed by administration every four weeks starting at Week 4 through Week 52 at the study site.

In accordance with the pre-specified prednisone taper (26 weeks for secukinumab arm; 52 weeks for placebo arm), participants will receive a combination of prednisone and secukinumab or prednisone and placebo from Baseline through Week 52.

Participants who do not achieve clinical remission by Week 12 or, experience a GCA clinical relapse after achieving clinical remission will enter Escape treatment. Participants in Escape treatment will receive GCs (prednisone or prednisone equivalent) at a dose as determined by the investigator in accordance with standard of care and continue to receive originally randomized double-blind study treatment. Alternatively, at the discretion of the investigator, participants could discontinue study treatment to receive Rescue treatment (alternative treatment for GCA).

Participants in either Escape or Rescue treatments will continue to attend all subsequent scheduled visits and assessments.
Treatment Period 2 (Week 56 dosing to Week 108): Starting at Week 56, in Treatment Period 2, participants will receive further treatment based on participant's clinical remission status as follows:
   Participants in sustained clinical remission at Week 56 (defined as participants who achieve clinical remission at Week 52 without a relapse through Week 56) will continue on study treatment as below through Treatment Period 2 ending at Week 108
   Participants initially randomized to secukinumab will be re-randomized in 1:1 ratio to secukinumab 300 mg s.c. q4w or placebo in double-blinded manner.
   Participants initially randomized to placebo will remain on placebo in a blinded manner.
   If participants have a clinical relapse during Treatment Period 2, they will be offered OL secukinumab 300 mg s.c. Treatment initiation of OL secukinumab will be similar to that for the secukinumab treatment in Treatment Period 1, administered every week (qw) for the first 4 weeks followed by administration every 4 weeks (q4w) starting 4 weeks after the first dose. GCs will be administered in accordance with standard of care with dosing determined by the investigator.
   Participants who have a clinical relapse and then do not achieve clinical remission by 12 weeks after starting OL secukinumab or have a clinical relapse on OL secukinumab during Treatment Period 2 will discontinue OL secukinumab and attend the EOT and EOS visits. Further treatment in accordance with standard of care will be determined by the investigator.
Participants not in sustained clinical remission at Week 56 (including also participants in Escape treatment) will be offered OL secukinumab 300 mg s.c. treatment starting at Week 56:
   Treatment initiation of OL secukinumab will be similar to that for the secukinumab treatment group, administered every week (qw) for the first 4 weeks followed by administration every 4 weeks (q4w) starting 4 weeks after the first dose. Participants will also receive GC treatment at a dose determined by the investigator in accordance with standard of care.
   Participants who do not achieve clinical remission by 12 weeks after starting OL secukinumab or have a clinical relapse on OL secukinumab during Treatment Period 2 will discontinue OL secukinumab and attend the EOT and EOS visits. Further treatment as determined by the investigator.

### Treatment Duration

The planned maximum duration of study treatment within this study is 108 weeks with last dose administration within the study at Week 104. Participants may be discontinued from study treatment earlier as noted above, or due to unacceptable toxicity, disease progression, and/or at the discretion of the investigator or the participant.

### Post-Trial Access

Participants who are on blinded study treatment (secukinumab or placebo) at Week 108: Participants who are on blinded study treatment and in sustained clinical remission at Week 108 (i.e., are only on study treatment, without concomitant GCs), will complete treatment (last dose at Week 104), complete the EOT visit at Week 108, and EOS visit at Week 116. For participants, who in the opinion of the investigator in accordance with standard practice, will derive clinical benefit from receiving secukinumab post-trial, every effort will be made to provide OL secukinumab in accordance with the local laws and regulations. For participants who start OL secukinumab following completion of study treatment, the EOS visit at Week 116 will be waived.
Participants who are on OL secukinumab at Week 108: Participants who are on OL secukinumab at Week 108 will complete treatment (last dose at Week 104), complete the EOT visit at Week 108, and EOS visit at Week 116. For participants who in the opinion of the investigator are still deriving clinical benefit from OL secukinumab, every effort will be made to continue provision of OL secukinumab in accordance with the local laws and regulations. If post-trial access to OL secukinumab is provided, the EOS visit at Week 116 will be waived. For participants who have not yet received 12 weeks of OL secukinumab, the investigator will assess if the participant is deriving clinical benefit from OL secukinumab and should continue treatment in post-trial access.

### Other Treatment(s)

### Concomitant Therapy

All medications, procedures, and significant non-drug therapies (including physical therapy and blood transfusions) administered after the participant was enrolled into the study must be recorded on the appropriate Case Report Form (CRF).

Each concomitant drug must be individually assessed against all exclusion criteria/prohibited medication. If in doubt, the investigator should contact the Novartis medical monitor before randomizing a participant or allowing a new medication to be started. If the participant is already enrolled, contact Novartis to determine if the participant should continue participation in the study.

Permitted Concomitant Therapy Requiring Caution and/or Action

### Methotrexate (MTX)

Participants taking MTX (≤25 mg/week) at study entry are allowed to continue their medication provided they have taken it for at least 3 months and are on a stable dose for at least 4 weeks prior to randomization and throughout the study.

Participants on MTX must be taking folic acid supplementation before randomization and during the trial to minimize the likelihood of MTX associated toxicity.

### Vitamin D

Vitamin D (at 1000 I.U. per day dose) and calcium supplements are strongly recommended during the study for osteoporosis prevention, given the use of corticosteroids in all treatment arms.

### REFERENCES

Cella D F, Tulsky D S, Gray G, et al (1993) The Functional Assessment of Cancer Therapy (FACT) scale: Development and validation of the general measure. Journal of Clinical Oncology; 11 (3):570-579.
Ciccia F, Rizzo A, Ferrante A, et al (2017) New insights into the pathogenesis of giant cell arteritis. Autoimmunity Reviews; 16(7):675-683.
De Silvia M and Hazleman B (1986) Azathioprine in giant cell arteritis polymyalgia rheumatica: a double-blind study. Ann Rheum Dis; 45(2):136-138.
Espigol-Frigole G, Corbera-Bellalta M, Planas-Rigol E, et al (2013) Increased IL17A expression in temporal artery lesions is a predictor of sustained response to glucocorticoid treatment in patients with giant-cell arteritis. Ann Rheum Dis; 72(9):1481-1487.
Hayat S (2008) Infliximab for Maintenance of Glucocorticosteroid-Induced Remission of Giant Cell Arteritis: A Randomized Trial. Yearbook of Medicine: 38-39.
Hoffmann G, Cid M, Hellmann D, et al (2002) A multicenter, randomized, double-blind, placebo-controlled trial of adjuvant methotrexate treatment for giant cell arteritis. Arthritis Rheum; 46(5):1309-1318.
ICH E2D (2004) Post Approval Safety Data Management: Note for guidance on definitions and standards for expedited reporting. Available from:
http://www.ema.europa.eu/docs/en_GB/document_library/Scientific_guideline/2009/09/WC500 002807.pdf (Accessed on 29 May 2018)
Jover J, Hernandez-Garcia C, Morado I, et al (2001) Combined treatment of giant-cell arteritis with methotrexate and prednisone. A randomized double-blind, placebo-controlled trial. Ann Intern Med; 134(2):106-114.
Koster M, Warrington J, Kermani T, et al (2016) Update on Epidemiology and Treatment of Giant Cell Arteritis. Current Treatment Options in Rheumatology; 2(2):138-152.
Marquez A, Hernandez-Rodriguez J, Cid M, et al (2014) Influence of the IL17A locus in giant cell arteritis susceptibility. Ann Rheum Dis; 73(9):1742-1745.
McClure N, Sayah F, Xie F, et al (2017) Instrument-Defined Estimates of the Minimally Important Difference for EQ-5D-5L Index Scores. Value Health; 20(4):644-650.
Miloslavsky E, Naden R, Bijlsma J, et al (2017) Development of a Glucocorticoid Toxicity Index (GTI) using multicriteria decision analysis. Annals of the Rheumatic Diseases; 76:543-546.
Miyabe C, Miyabe Y, Strle K, et al (2017) An expanded population of pathogenic regulatory T cells in giant cell arteritis is abrogated by IL-6 blockade therapy. Ann Rheum Dis; 76:898-905.
Mukhtyar C, Guillevin, L, Cid M, et al (2009) EULAR recommendations for the management of large vessel vasculitis. Ann Rheum Dis; 68:318-323.
Ness T, Bley T, Schmidt W, et al (2013) The diagnosis and treatment of giant cell arteritis. Dtsch Arztebl Int; 110(21):376-385.
Petri H, Nevitt A, Sarsour K, et al (2014) Incidence of giant cell arteritis and characteristics of patients: data-driven analysis of cormorbidities. Arthritis Gare Res (Hoboken); 67(3):390-395.
Proven A, Gabriel S E, Orces C, et al (2003) Glucocorticoid therapy in giant cell arteritis: Duration and adverse outcomes. Arthritis & Rheumatism 2003; 49(5):703-708.
Reichenbach S, Adler S, Bonel H, et al (2018) Magnetic resonance angiography in giant cell arteritis: results of a randomized controlled trial of tocilizumab in giant cell arteresis. Rheumatology; 1: 57(6):982-986.
Rotar Z, Tomsic M, Hocevar A, et al (2018) Secukinumab for the maintenance of glucocorticoid-free remission in a patient with giant cell arteritis and psoriatic arthritis. Rheumatology; 57(5):934-936.
Salvarani C, Cantini F, Boiardi L, et al (2004) Polymyalgia rheumatica. Best Pract Res Clin Rheumaol; 18(5):705-722.
Samson M, Audia S, Fraszczak J, et al (2012) Th1 and Th17 lympocytes expressing CD 16 are implicated in giant cell arteritis and polymyalgia rheumatica pathogenesis. Arthritis Rheum; 64(11):3788-3798.
Samson M, Corbera-Bellalta M, Audia S, et al (2017) Recent advances in our understanding of giant cell arteritis pathogenesis. Autoimmunity Reviews; 16(8):833-844.
Sammut L, Litwic A, Smith R, et al (2018) 002 Biopsy proven giant cell arteritis in a patient with psoriatic arthritis on TNF-alpha inhibitor treated with high dose prednisolone and a switch to secukinumab (anti IL-17). Rheumatology, Volume 57, Issue suppl_3. Available at [https://doi.org/10.1093/rheumatology/key075.226]
Stone J, Tuckwell K, Dimonaco S, et al (2017) Trial of Tocilizumab in Giant-Cell Arteritis. The New England Journal of Medicine. N Engl J Med; 377:317-328.
Tfelt-Hansen P, Pascual J, Ramadan N, et al [International Headache Society Clinical Trials Subcommittee members] (2012) Guidelines for controlled trials of drugs in migraine: Third edition. A guide for investigators. Cephalalgia; 32(1):6-38.
Ware J, Kosinki M, Gandeck B (1993) SF-36 Health Survey manual and interpretation guide. Boston: The Health Institute, New England Medical Center.
Ware J, Kosinki M, Gandeck B (1994) SF-36 Health Survey manual and interpretation guide. Update. Boston: The Health Institute, New England Medical Center.
Xu R, Insinga R, Golden W, et al (2011) EuroQol (EQ-5D) health utility scores for patients with migraine. Qual Life Res; 20(4):601-8.
Yellen S B, Cella D F, Webster K, et al (1997) Measuring Fatigue and Other Anemia-Related Symptoms with the Functional Assessment of Cancer Therapy (FACT) Measurement System. Journal of Pain and Symptom Management; 13:63-74.

1. A method of treating giant cell arteritis (GCA), comprising subcutaneously administering to a patient in need thereof about 150 mg-about 300 mg of an IL-17 antibody or antigen-binding fragment thereof, wherein the IL-17 antibody or antigen-binding fragment thereof binds to an epitope of a human IL-17 homodimer having two mature human IL-17 protein chains, said epitope comprising Leu74, Tyr85, His86, Met87, Asn88, Val124, Thr125, Pro126, Ile127, Val128, and His129 on one chain and Tyr43, Tyr44, Arg46, Ala79, and Asp80 on the other chain, wherein the IL-17 antibody or antigen-binding fragment thereof has a K_{D} for human IL-17 of about 100-200 pM, and wherein the IL-17 antibody or antigen-binding fragment thereof has an in vivo half-life of about 4 weeks.

2. The method of claim 1, wherein the patient is administered the IL-17 antibody or antigen-binding fragment thereof as a loading dose.

3. The method of claim 1, wherein the patient is administered the IL-17 antibody or antigen-binding fragment thereof as a weekly loading dose.

4. The method of claim 3, wherein the patient is administered the IL-17 antibody or antigen-binding fragment thereof during week 0, 1, 2, 3, and 4.

5. The method of claim 1, wherein the patient is administered the IL-17 antibody or antigen-binding fragment thereof every two weeks or every four weeks.

6. The method of claim 5, wherein the patient is administered the IL-17 antibody or antigen-binding fragment thereof for a total treatment duration of at least 26 weeks, or at least 52 weeks, or at least 2 years.

7. The method of claim 3, wherein the patient is administered the IL-17 antibody or antigen-binding fragment thereof as a weekly loading dose during week 0, 1, 2, 3, and 4 and every two weeks thereafter or every four weeks thereafter.

8. The method claim 1, wherein the patient is administered the IL-17 antibody or antigen-binding fragment thereof weekly during week 0, 1, 2, 3, and 4, and then every 4 weeks.

9. The method of claim 8, wherein the patient is administered the IL-17 antibody or antigen-binding fragment thereof during week 0, 1, 2, 3, 4, 8 and 12.

10. The method of claim 8, wherein the patient is administered the IL-17 antibody or antigen-binding fragment thereof weekly during week 0, 1, 2, 3, and 4, and then every 4 weeks thereafter, for a total treatment duration of at least 26 weeks, at least 52 weeks, or at least 2 years.

11. The method of claim 1, wherein, prior to treatment with the IL-17 antibody or antigen-binding fragment thereof, the patient failed to respond to, had an inadequate response to, or was intolerant to a prior GCA treatment selected from the group consisting of treatment with a corticosteroid, such as prednisone, prednisolone, or methylprednisolone, treatment with a TNF-alpha inhibitor, treatment with an IL-6 inhibitor, treatment with methotrexate, and combinations thereof.

12. The method of claim 1, wherein treatment with the IL-17 antibody or antigen-binding fragment thereof reduces the dose of corticosteroid sufficient to induce or maintain effective GCA treatment or GCA remission.

13. The method of claim 1, wherein the method comprises therapeutically effective treatment of GCA without a corticosteroid; and/or wherein the method induces or maintains GCA remission without concomitant corticosteroid treatment.

14. The method of claim 13, wherein treatment with the IL-17 antibody or antigen-binding fragment thereof increases the time to flare in the patient, wherein a flare is indicated by signs or symptoms of GCA and/or erythrocyte sedimentation rate (ESR)≥30 mm/hr and/or CRP≥10 mg/L.

15. The method of claim 1, wherein the patient has a reduced cumulative corticosteroid dose after 28 weeks, or 52 weeks, or 2 years of treatment with the IL-17 antagonist, as compared to a patient undergoing treatment according to a standard of care.

16. (canceled)

17. The method of claim 1, wherein the patient is effectively treated for GCA with the IL-17 antibody or antigen-binding fragment thereof and a corticosteroid dose equivalent to ≤5 mg/day of prednisolone.

18. The method of claim 17, wherein treatment with the IL-17 antibody or antigen-binding fragment thereof induces improvement in one or more clinical assessments selected from the group consisting of Physician's global assessment (PhGA) visual analog scale (VAS); Patient reported outcomes (PROs); Patient global assessment (PGA) VAS; Functional Assessment of Chronic Illness Therapy Fatigue (FACIT-Fatigue); Short form 36 (SF36); and EuroQoL 5D (EQ-5D); and/or wherein the patient exhibits a reduction in vessel inflammation by ultrasound and/or MRI, e.g., wherein the reduction in vessel inflammation is greater than achieved in the absence of treatment with the IL-17 antibody or antigen-binding fragment thereof, greater than achieved by treatment with corticosteroid alone or in combination with an IL-6 or TNF-alpha inhibitor, or greater than achieved by treatment with an IL-6 or TNF-alpha inhibitor.

19. The method of claim 1, wherein the IL-17 antibody or antigen-binding fragment thereof comprises:
i) an immunoglobulin heavy chain variable domain (V_{H}) comprising the amino acid sequence set forth as SEQ ID NO:8;
ii) an immunoglobulin light chain variable domain (V_{L}) comprising the amino acid sequence set forth as SEQ ID NO:10;
iii) an immunoglobulin V_{H} domain comprising the amino acid sequence set forth as SEQ ID NO: 8 and an immunoglobulin V_{L} domain comprising the amino acid sequence set forth as SEQ ID NO:10;
iv) an immunoglobulin V_{H} domain comprising the hypervariable regions set forth as SEQ ID NO:1, SEQ ID NO:2, and SEQ ID NO:3;
v) an immunoglobulin V_{L} domain comprising the hypervariable regions set forth as SEQ ID NO: 4, SEQ ID NO:5 and SEQ ID NO:6;
vi) an immunoglobulin V_{H} domain comprising the hypervariable regions set forth as SEQ ID NO: 11, SEQ ID NO:12 and SEQ ID NO:13;
vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6;
viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO: 6;
ix) an immunoglobulin light chain comprising the amino acid sequence set forth as SEQ ID NO: 14;
x) an immunoglobulin heavy chain comprising the amino acid sequence set forth as SEQ ID NO: 15; or
xi) an immunoglobulin light chain comprising the amino acid sequence set forth as SEQ ID NO: 14 and an immunoglobulin heavy chain comprising the amino acid sequence set forth as SEQ ID NO:15.

20. The method of claim 19, wherein the IL-17 antibody or antigen-binding fragment thereof is secukinumab.

21. A method of treating a patient having active GCA, comprising administering to the patient about 300 mg of secukinumab by subcutaneous injection at weeks 0, 1, 2, 3, and 4, and then every four weeks thereafter.

22. A method of treating a patient having active GCA, comprising administering to the patient about 300 mg of secukinumab by subcutaneous injection at weeks 0, 1, 2, 3, and 4, and then every four weeks thereafter, for a total treatment duration of at least 52 weeks.

23. (canceled)

24. The method of claim 1, wherein prior to the treatment, the patient has active GCA as indicated by Unequivocal cranial symptoms of GCA; Temporal artery biopsy (TAB) revealing features of GCA and/or cross-sectional imaging study such as ultrasound (e.g. cranial or axillary), MRA, CTA, or PET-CT with evidence of vasculitis.

25. The method of claim 1, wherein prior to the treatment the patient has elevated erythrocyte sedimentation rate (ESR)≥30 mm/hr or C-reactive protein (CRP)≥10 mg/L attributed to active GCA or active GCA on TAB or imaging study.

26. (canceled)

27. (canceled)

28. The method of claim 20, wherein the method comprises administering a loading dose of 150 mg secukinumab once a week at weeks 0, 1, 2, 3, and 4, and thereafter administering a maintenance dose of 150 mg secukinumab monthly, every four weeks, or every two weeks.

29. (canceled)

30. (canceled)

31. The method of claim 20, wherein the method comprises administering a loading dose of 300 mg secukinumab once a week at weeks 0, 1, 2, 3, and 4, and thereafter administering a maintenance dose of 300 mg secukinumab monthly, every four weeks, or every two weeks.

32. (canceled)

33. (canceled)


SEQUENCE LISTING (31 sequences)

SEQ ID NO: 1 - protein, 5 aa, synthetic construct
NYWMN

SEQ ID NO: 2 - protein, 17 aa, synthetic construct
AINQDGSEKYYVGSVKG

SEQ ID NO: 3 - protein, 18 aa, synthetic construct
DYYDILTDYYIHYWYFDL

SEQ ID NO: 4 - protein, 12 aa, synthetic construct
RASQSVSSSYLA

SEQ ID NO: 5 - protein, 7 aa, synthetic construct
GASSRAT

SEQ ID NO: 6 - protein, 9 aa, synthetic construct
QQYGSSPCT

SEQ ID NO: 7 - DNA, 381 bp, Homo sapiens
  source 1..381: mol_type = genomic DNA
  CDS 1..381: protein_id = 8
gaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctca

SEQ ID NO: 8 - protein, 127 aa, Homo sapiens [sequence omitted]

SEQ ID NO: 9 - DNA, 327 bp, Homo sapiens
  source 1..327: mol_type = genomic DNA
  CDS 1..327: protein_id = 10; translation = EIVLTQSPGTLSLSPGERATLSCRASQSVSSSYLAWYQQKPGQA PRLLIYGASSRATGIPDRFSGSGSGTDFTLTISRLEPEDFAVYYCQQYGSSPCTFGQG TRLEIKR
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacga

SEQ ID NO: 10 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 11 - protein, 10 aa, synthetic construct
GFTFSNYWMN

SEQ ID NO: 12 - protein, 11 aa, synthetic construct
AINQDGSEKYY

SEQ ID NO: 13 - protein, 23 aa, synthetic construct
CVRDYYDILTDYYIHYWYFDLWG

SEQ ID NO: 14 - protein, 215 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 457 aa, Homo sapiens
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 11 aa, Homo sapiens
RPSQGINWELA

SEQ ID NO: 17 - protein, 7 aa, Homo sapiens
SQGINWE

SEQ ID NO: 18 - protein, 7 aa, Homo sapiens
DASSLEQ

SEQ ID NO: 19 - skipped

SEQ ID NO: 20 - protein, 9 aa, Homo sapiens
QQFNSYPLT

SEQ ID NO: 21 - protein, 6 aa, Homo sapiens
FNSYPL

SEQ ID NO: 22 - protein, 107 aa, Homo sapiens
AIQLTQSPSSLSASVGDRVTITCRPSQGINWELAWYQQKPGKAPKLLIYDASSLEQGVPSRFSGSGSGTDFTLTISSLQPEDFATYYCQQFNSYPLTFGGGTKVEIK

SEQ ID NO: 23 - protein, 214 aa, Homo sapiens
AIQLTQSPSSLSASVGDRVTITCRPSQGINWELAWYQQKPGKAPKLLIYDASSLEQGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 5 aa, Homo sapiens
SYWMS

SEQ ID NO: 25 - protein, 7 aa, Homo sapiens
GFTFSSY

SEQ ID NO: 26 - protein, 16 aa, Homo sapiens
NIKQDGSEKYYVDSVK

SEQ ID NO: 27 - protein, 6 aa, Homo sapiens
KQDGSE

SEQ ID NO: 28 - protein, 7 aa, Homo sapiens
DRGSLYY

SEQ ID NO: 29 - protein, 7 aa, Homo sapiens
DRGSLYY

SEQ ID NO: 30 - protein, 116 aa, Homo sapiens
EVQLVESGGDLVQPGGSLRLSCAASGFTFSSYWMSWVRQAPGKGLEWVANIKQDGSEKYYVDSVKGRFTISRDNAKNSLYLQMNSLRAEDTAVYYCARDRGSLYYWGQGTLVTVSS

SEQ ID NO: 31 - protein, 446 aa, Homo sapiens
EVQLVESGGDLVQPGGSLRLSCAASGFTFSSYWMSWVRQAPGKGLEWVANIKQDGSEKYYVDSVKGRFTISRDNAKNSLYLQMNSLRAEDTAVYYCARDRGSLYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK